(19) United States
(12) Patent Application Publication
(10) Pub. No.: US 2022/0249457 A1
(43) Pub. Date: Aug. 11, 2022

(54) METHOD OF MODULATING RIBONUCLEOTIDE REDUCTASE

(71) Applicants: CASE WESTERN RESERVE UNIVERSITY, Cleveland, OH (US); TAIPEI MEDICAL UNIVERSITY, Taipei (TW)

(72) Inventors: Chris DEALWIS, Cleveland, OH (US); Hsueh-Yun LEE, Taipei (TW); Rajesh VISWANATHAN, Cleveland, OH (US); Sarah HUFF, Cleveland, OH (US)

(21) Appl. No.: 17/728,623

(22) Filed: Apr. 25, 2022

Related U.S. Application Data
(63) Continuation-in-part of application No. PCT/US20/57080, filed on Oct. 23, 2020.
(60) Provisional application No. 62/925,092, filed on Oct. 23, 2019.

(51) Int. Cl. A61K 31/4439; A61K 31/4245; A61K 31/444; A61K 31/41; A61K 31/4192

(52) U.S. Cl. CPC A61K 31/4439; A61K 31/4245; A61K 31/4192; A61K 31/41; A61K 31/444

(57) ABSTRACT

A method of modulating ribonucleotide reductase activity in a neoplastic cell includes administering to the cell an amount of an oxadiazole, thiazole, diazole, triazole, or tetrazole ribonucleotide reductase modulator (RRmod), the amount being effective to inhibit neoplastic cell growth.

### RELATED APPLICATION

This application is a Continuation-in-Part of PCT/US2020/057080, filed Oct. 23, 2020, and claims priority from U.S. Provisional Application No. 62/925,092, filed Oct. 23, 2019, the subject matter of which are incorporated herein by reference in their entirety.

### GOVERNMENT FUNDING

This invention was made with government support under GM100887 and CA100827 awarded by The National Institutes of Health. The government has certain rights in the invention.

### FIELD OF THE INVENTION

This application relates to selective oxadiazole, thiazole, diazole, triazole, and tetrazole modulators of ribonucleotide reductase (RR) and to methods of using such modulators for therapeutic applications.

### BACKGROUND

Ribonucleotide reductase (RR) is a highly regulated enzyme which catalyzes the de novo dNTP synthesis pathway that is ubiquitously present in human, bacteria, yeast, and other organisms. RR plays a crucial role in de novo DNA synthesis by reducing ribonucleoside diphosphates to 2′-deoxy ribonucleoside diphosphates and maintains balanced pools of deoxynucleoside triphosphates (dNTPs) in the cell.

RRs are divided into three classes, I to III, based on the method of free-radical generation. All eukaryotic organisms encode a class I RR, consisting of an αnβn multi-subunit protein complex, in which the minimally active form is α2β2. The α or RR1 (large) subunit contains the catalytic (C-site) and two allosteric sites, while the β or RR2 subunit houses a stable tyrosyl free radical that is transferred some 35 Å to the catalytic site to initiate radical-based chemistry on the substrate.

RR is regulated transcriptionally, allosterically and, in the yeast S. cerevisiae, RR is further regulated by subunit localization and by its protein inhibitor Sml1. In mammalian cells, RR activity is also controlled by the RR2 levels. Consistent with the varying RR2 levels, dNTP pools also vary with the phases of the cell cycle, reaching the highest concentration during S-phase. RR is regulated by an intricate allosteric mechanism. The two previously described allosteric sites of RR are the specificity site (S-site), which determines substrate preference, and the activity site (A-site), which stimulates or inhibits RR activity depending on whether ATP or dATP is bound.

RR is directly involved in neoplastic tumor growth, metastasis, and drug resistance. The proliferation of cancer cells requires excess dNTPs for DNA synthesis. Therefore, an increase in RR activity is necessary as it helps provide extra dNTPs for DNA replication in primary and metastatic cancer cells. Because of this critical role in DNA synthesis, RR represents an important target for cancer therapy. However, existing chemotherapies that target ribonucleotide reductase are nucleoside-based analogs. Hence, they are promiscuous, leading to nonspecific binding of other nucleoside binding proteins which results in unwanted side effects. Therefore, there is a need for compositions and methods for specifically targeting and inhibiting RR activity in neoplastic cells in the treatment of neoplastic disorders.

### SUMMARY

Embodiments described herein relate to compounds and methods of modulating ribonucleotide reductase activity in a neoplastic cell. In some embodiments, the method can include administering to a neoplastic cell an amount of a ribonucleotide reductase modulator (RRmod) effective to inhibit neoplastic cell growth.

In some embodiments, the RRmod can include a compound having the following formula (I):

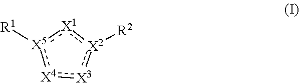

or a pharmaceutically acceptable salt, tautomer, or solvate thereof;

wherein:

X¹, X³, and X⁴ are each independently C, S, O, or N;

X² and X⁵ are each independently C or N;

at least two but no more than four of X¹, X², X³, X⁴, or X⁵ is not C;

---- is an optional bond;

R¹ and R² are each independently an alkyl, aryl, heteroaryl, or heterocyclyl optionally substituted with one or more R⁴; and

each R⁴ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety, and wherein R¹ and R² are not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define an oxadiazole, thiazole, diazole, triazole, or tetrazole.

In some embodiments, each R⁴ is independently halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R¹ and R² are not benzoic acid.

In other embodiments, R¹ and R² are each independently selected from the group consisting of:

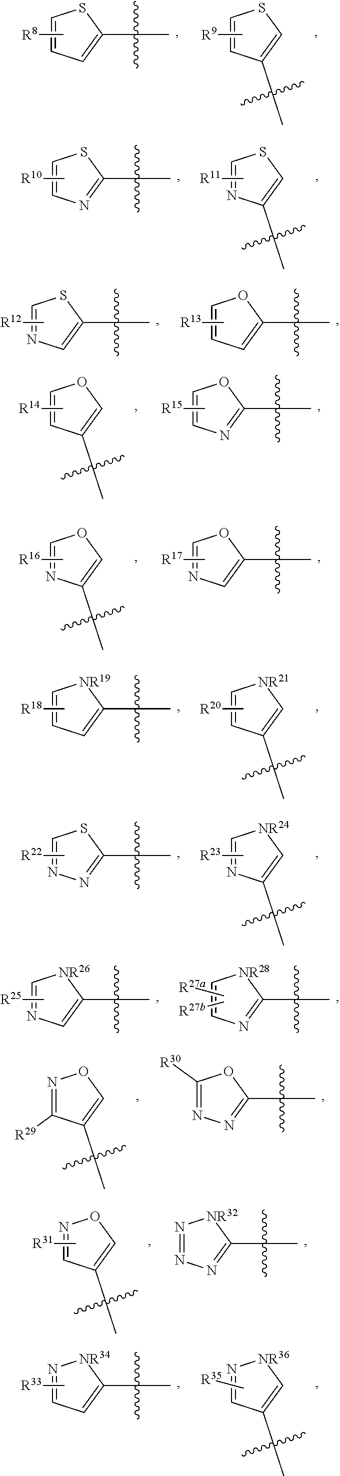

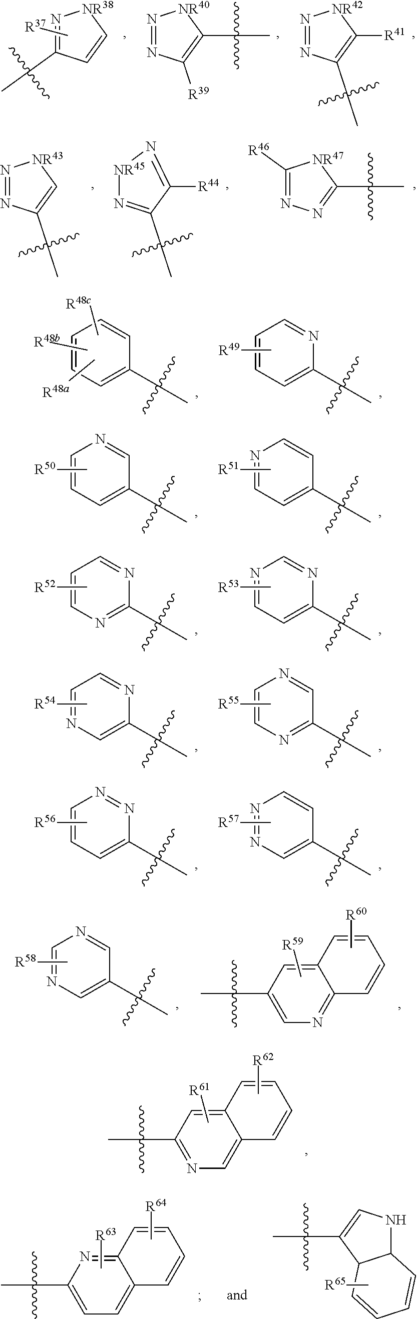

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R¹ and R² are each independently a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

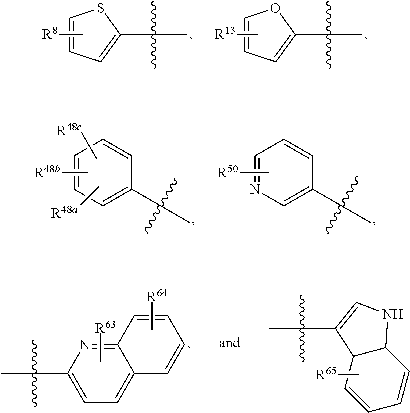

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In still other embodiments, the compound or RRmod can have a molecular weight (g/mol) less than about 400 g/ml, less than about 390 g/mol, less than about 380 g/mol, less than about 370 g/mol less than about 360 g/mol, less than about 350 g/mol, less than about 340 g/mol, less than about 330 g/mol, less than about 320 g/mol, less than about 310 g/mol, less than about 300 g/mol, less than about 290 g/mol, less than about 280 g/mol, less than about 270 g/mol, or less than about 260 g/mol. For example, the compound can have molecular weight (g/mol) of about 230 g/mol to about 400 g/mol, about 240 g/mol to about 360 g/mol or about 250 g/mol to about 350 g/mol.

In some embodiments, the compound or RRmod can inhibit the enzymatic activity of recombinant human ribonucleotide reductase at an IC₅₀ of less than 10 μM, at an IC₅₀ of less than 5 μM, at an IC₅₀ of less than 1 μM, at an IC₅₀ of less than 250 nM, at an IC₅₀ of less than 50 nM, at an IC₅₀ of less than 10 nM, at an IC₅₀ of less than 5 nM at a recombinant, at an IC₅₀ of about 2.5 nM to about 10 nM, or less than about 2.5 nM.

In some embodiments, the RRmod can include a compound having the following formula (II):

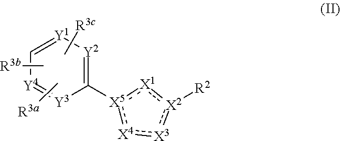

or a pharmaceutically acceptable salt, tautomer, or solvate thereof;

wherein:

X¹, X³, and X⁴ are each independently C, S, O, or N;

X² and X⁵ are each independently C or N;

at least two but no more than four of X¹, X², X³, X⁴, or X⁵ is not C;

Y¹, Y², Y³, Y⁴ are each independently N or C, provided no more than 3 of Y¹, Y², Y³, or Y⁴ are N;

---- is an optional bond;

R² is an alkyl, aryl, heteroaryl, or heterocyclyl optionally substituted with one or more R⁴;

R^{3a}, R^{3b}, R^{3c}, and each R⁴ are each independently absent, halogen, or optionally substituted hydroxyl, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, heterocyclyl, alkylaryl, aromatic moiety, or heteroaromatic moiety, and R² is not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define an oxadiazole, thiazole, diazole, triazole, or tetrazole.

In some embodiments, R^{3a}, R^{3b}, and R^{3c} are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R² is not benzoic acid.

In some embodiments, R² is independently selected from the group consisting of:

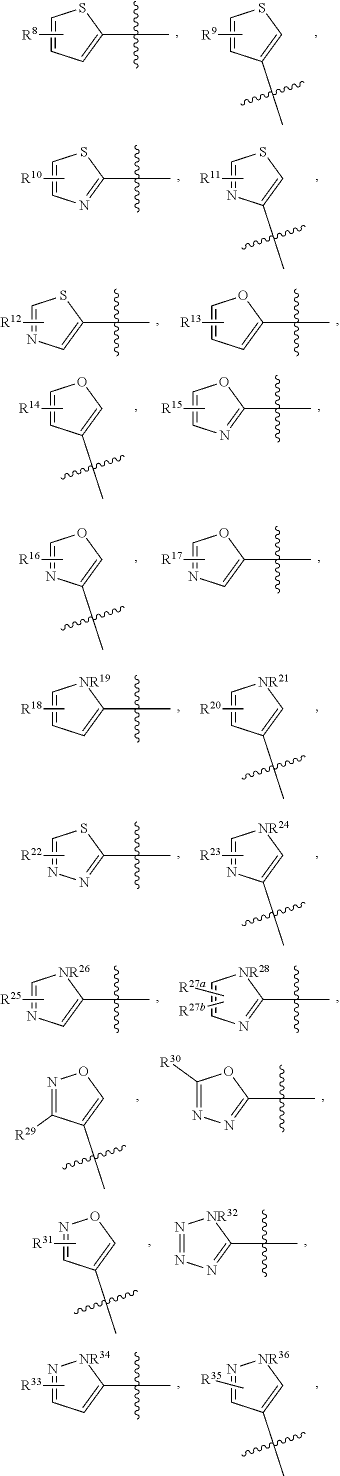

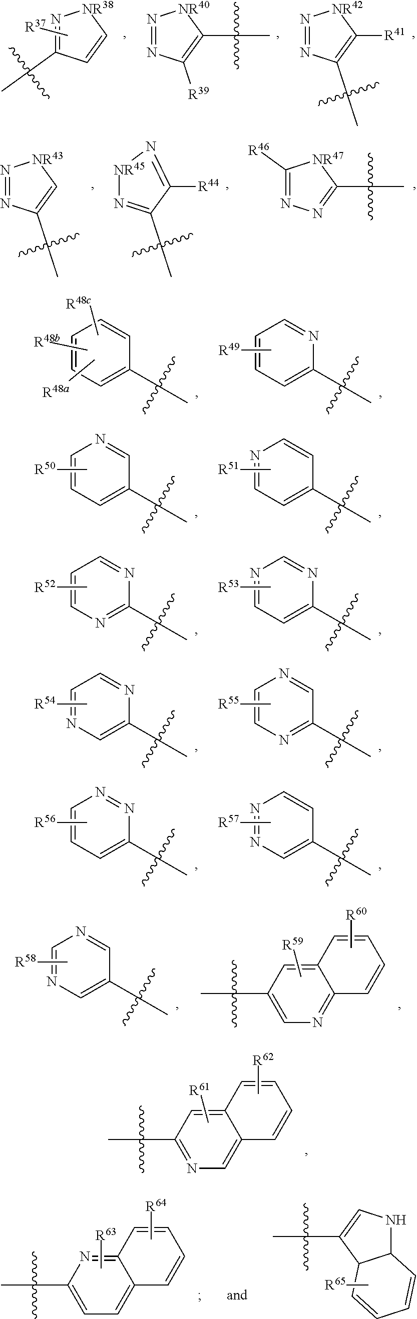

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

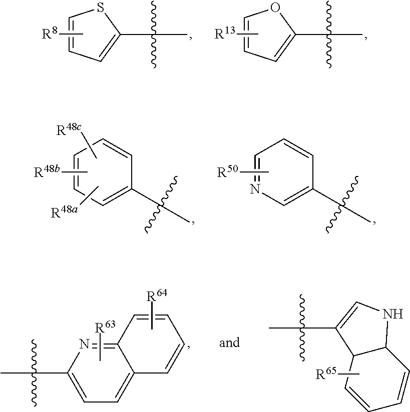

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, the RRmod can include a compound having the following formula (III):

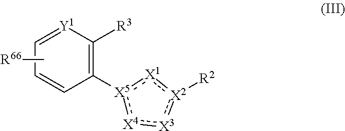

or a pharmaceutically acceptable salt, tautomer, or solvate thereof, wherein:

wherein:

X¹, X³, and X⁴ are each independently C, S, O, or N;

X² and X⁵ are each independently C or N;

at least two but no more than four of X¹, X², X³, X⁴, or X⁵ is not C;

Y¹ is Nor C;

---- is an optional bond;

R² is an alkyl, aryl, heteroaryl, or heterocyclyl optionally substituted with one or more R⁴;

R³ and R⁴ are each independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;

R⁶⁶ is absent, halogen, alkoxy, or amino; and

R² is not a naphtholyl.

In some embodiments, at least one of X¹, X³, or X⁴ is N. For example, one, two, or three of X¹, X³, or X⁴ is N.

In other embodiments, at least one of X² or V is C.

In some embodiments, X¹, X², X³, X⁴, and X⁵ define an oxadiazole, thiazole, diazole, triazole, or tetrazole.

In some embodiments, R³ is halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R² is not benzoic acid.

In some embodiments, R² is independently selected from the group consisting of:

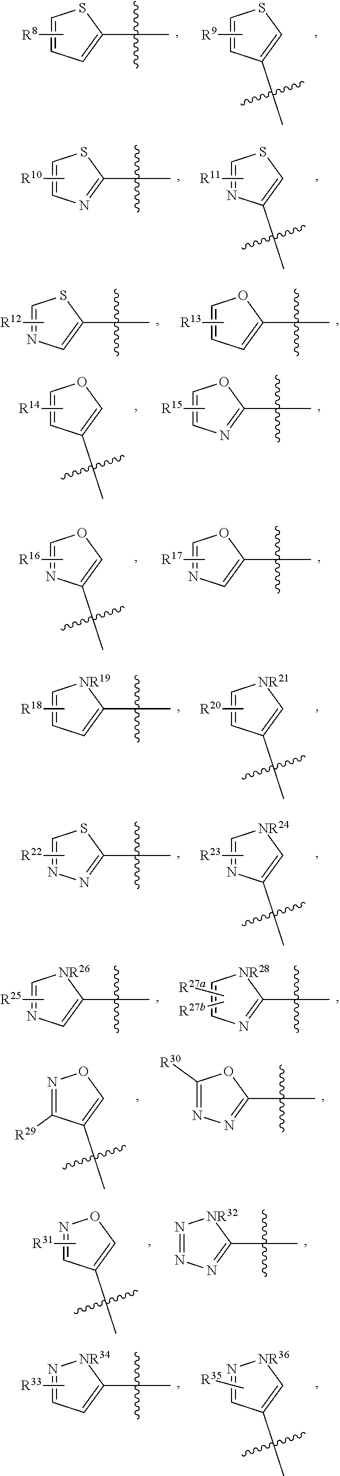

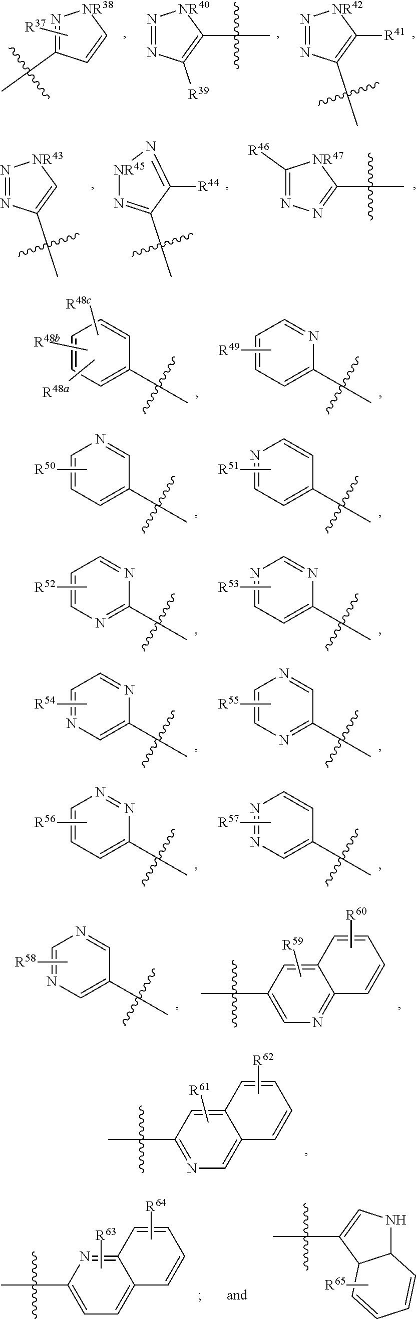

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

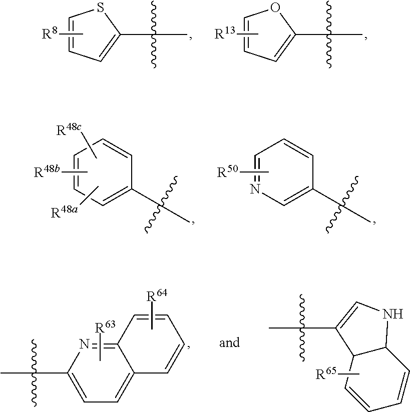

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, the RRmod can include a compound having the following formula (IV):

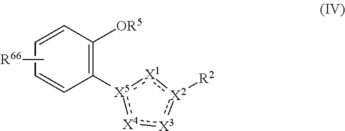

or a pharmaceutically acceptable salt, tautomer, or solvate thereof, wherein:

wherein:

X¹, X³, and X⁴ are each independently C, S, O, or N;

X² and X⁵ are each independently C or N;

at least two but no more than four of X¹, X², X³, X⁴, or X⁵ is not C;

---- is an optional bond;

R² is an alkyl, aryl, heteroaryl, or heterocyclyl optionally substituted with one or more R⁴;

each R⁴ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety,

R⁵ is H, C₁-C₆ alkyl, aryl, or —(CO)-alkyl;

R⁶⁶ is absent, halogen, alkoxy, or amino; and

R² is not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define an oxadiazole, thiazole, diazole, triazole, or tetrazole.

In some embodiments, R² is not benzoic acid.

In some embodiments, R² is independently selected from the group consisting of:

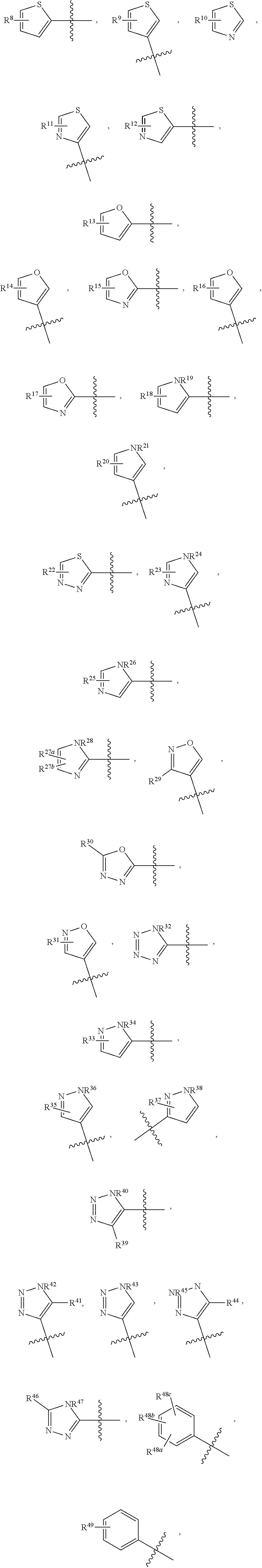

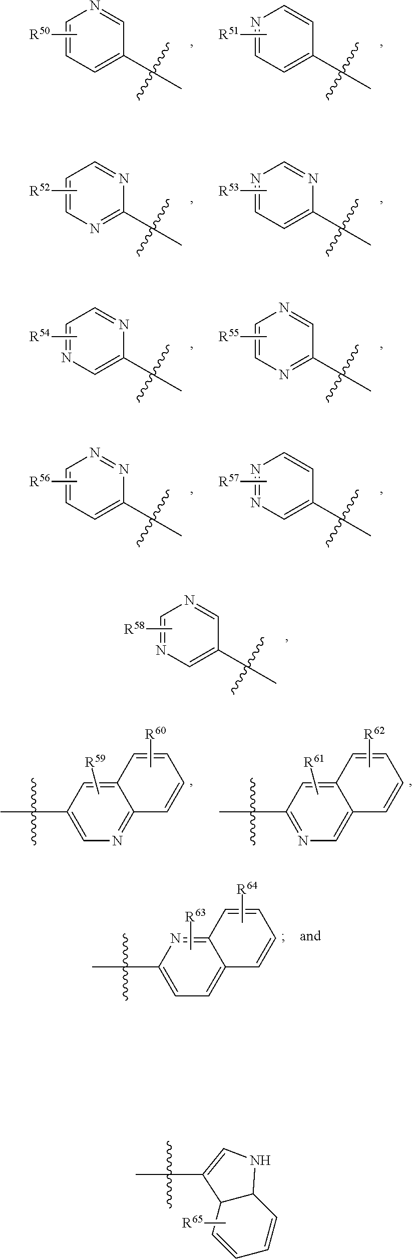

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

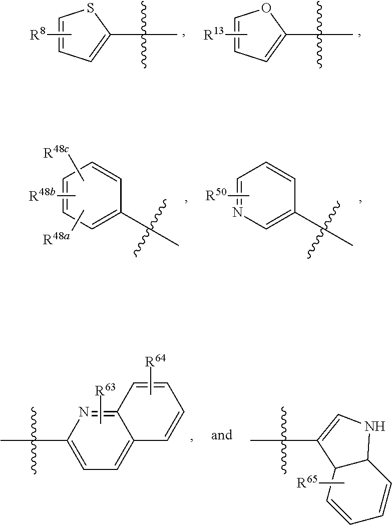

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, the RRmod can include a compound having the following formula (V):

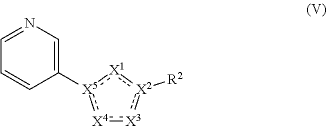

or a pharmaceutically acceptable salt, tautomer, or solvate thereof, wherein:

X¹, X², and X³ are each independently N or O, and two of X¹, X², and X³ are N;

---- is an optional bond;

R² is an aryl group or heteroaryl group optionally substituted with one or more R³; and

each R³ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety, and wherein R² is not a naphthol group.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define oxadiazole, thiazole, diazole, triazole, or tetrazole.

In some embodiments, R² is not benzoic acid.

In some embodiments, R² is independently selected from the group consisting of:

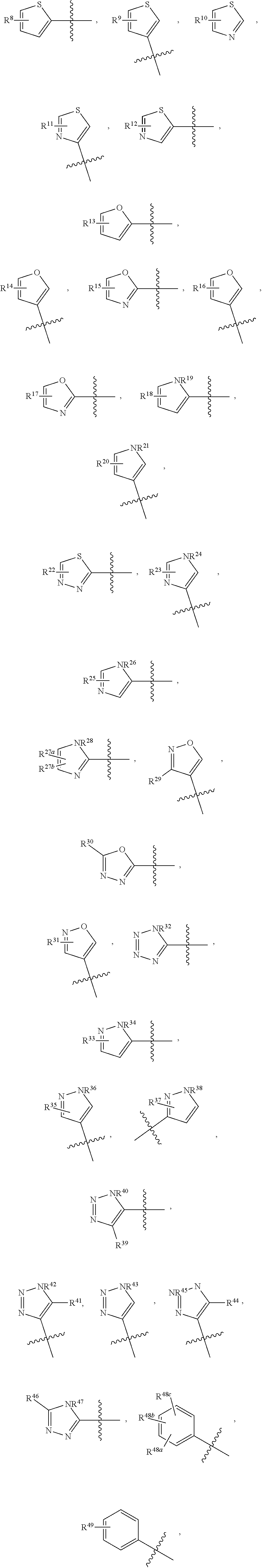

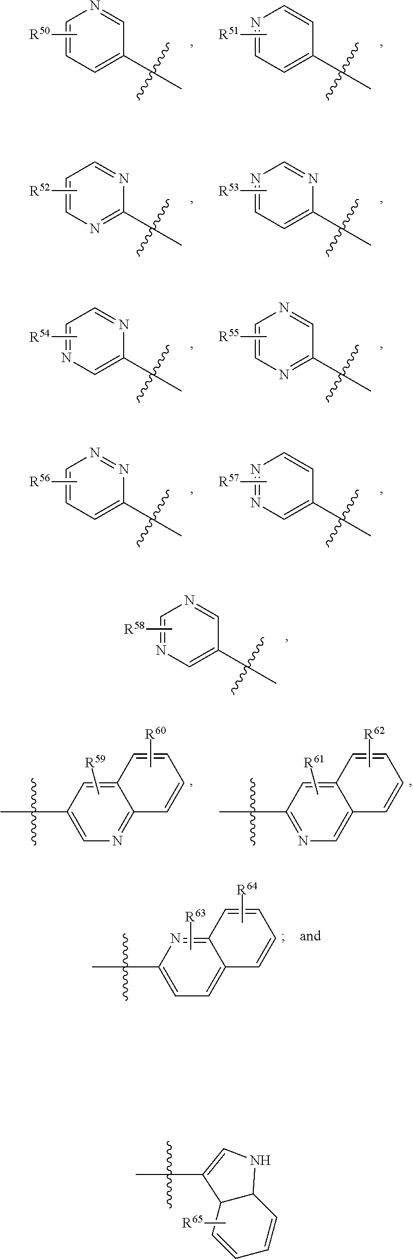

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In other embodiments, an RRmod can be a compound selected from:

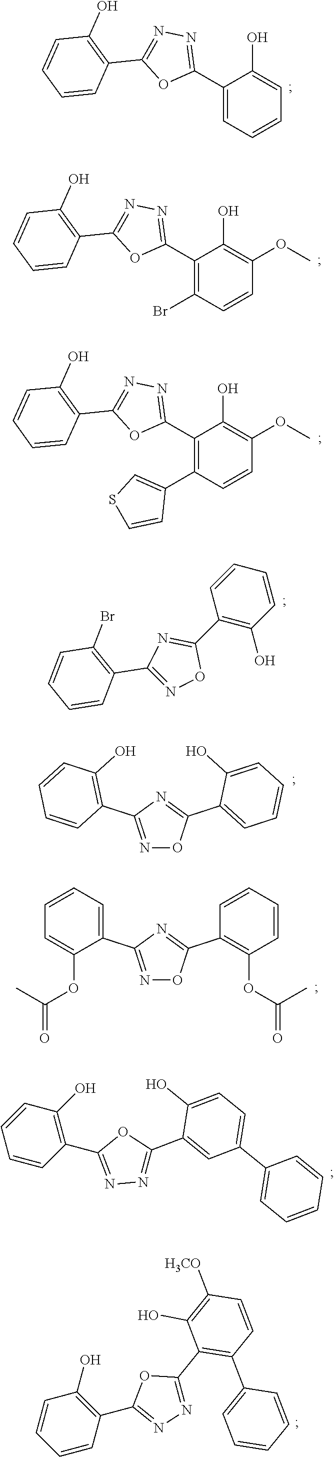

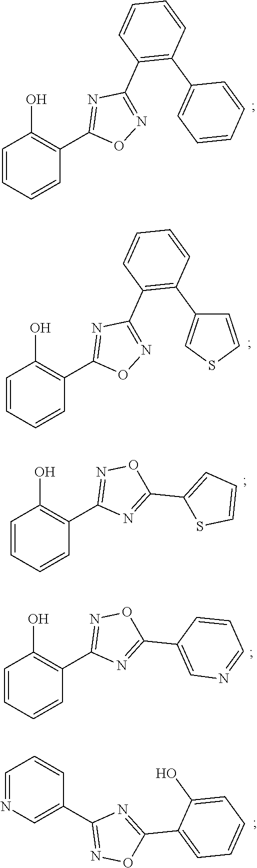

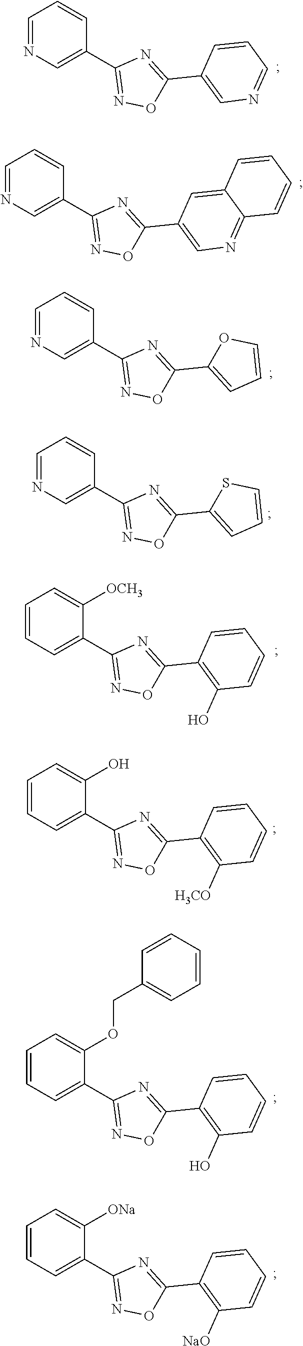

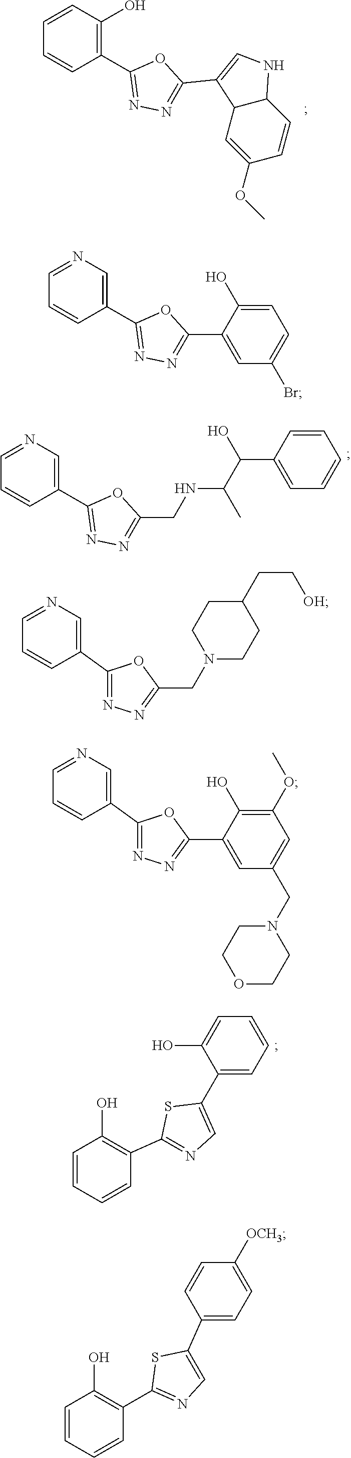

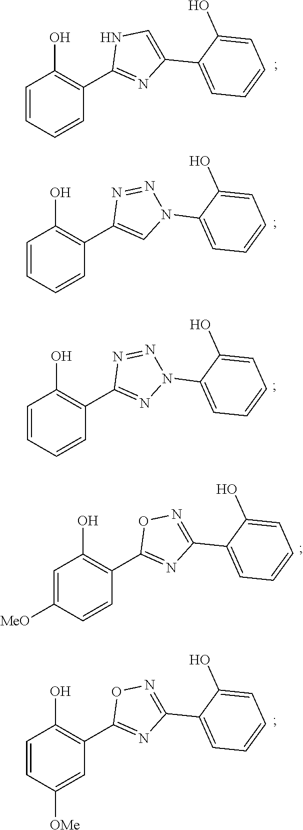 [SMILES: Oc1ccccc1-c1c[nH]c(-c2ccccc2O)n1 ; Oc1ccccc1-c1cn(-c2ccccc2O)nn1 ; Oc1ccccc1-c1nnn(-c2ccccc2O)n1 ; COc1ccc(-c2nc(-c3ccccc3O)no2)c(O)c1 ; COc1ccc(O)c(-c2nc(-c3ccccc3O)no2)c1]

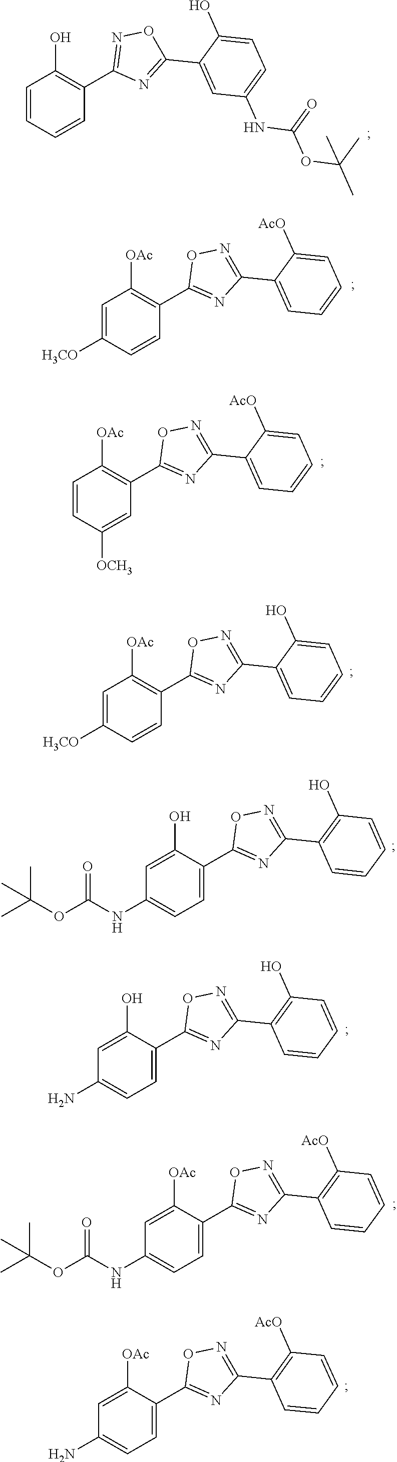

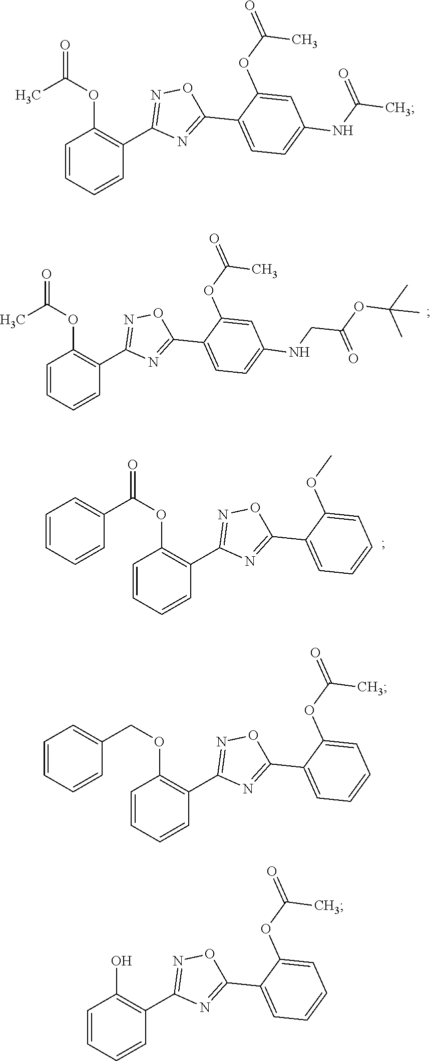 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]

a halogen (e.g., F, Cl, Br) substituted compound thereof; or a pharmaceutically acceptable salt, tautomer, or solvate thereof.

In other embodiments, an RRmod can be an oxadiazole selected from:

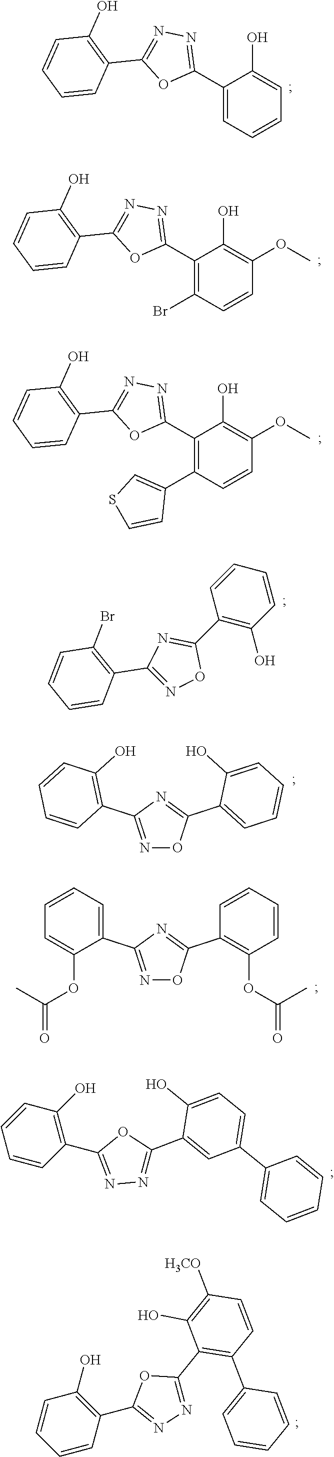

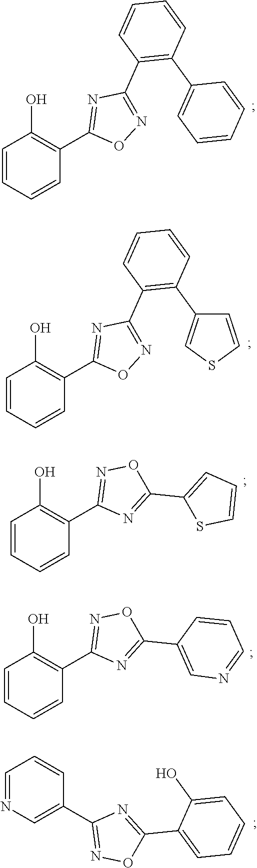

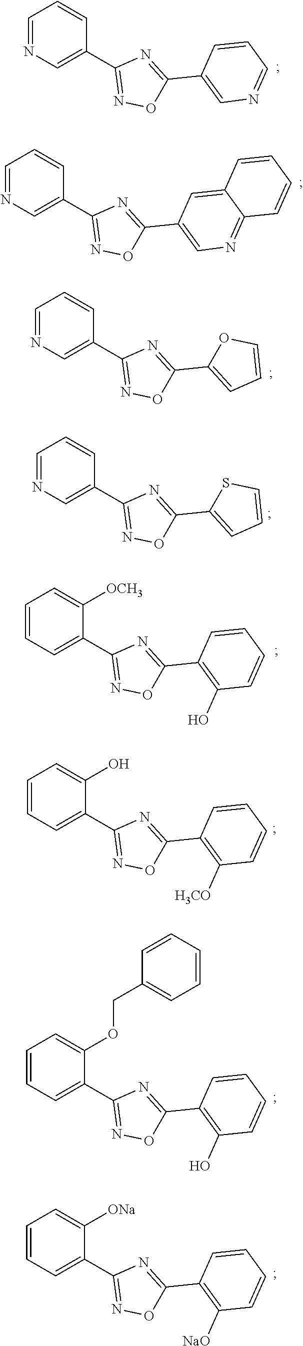

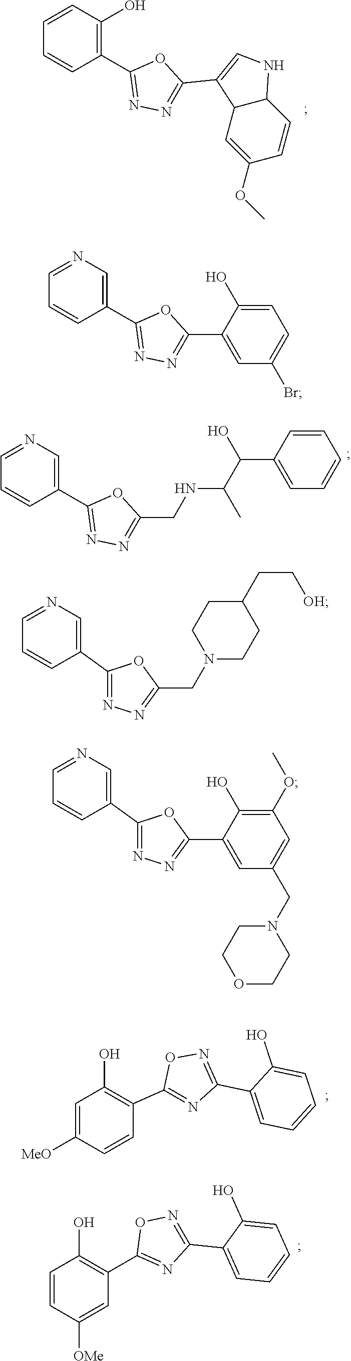

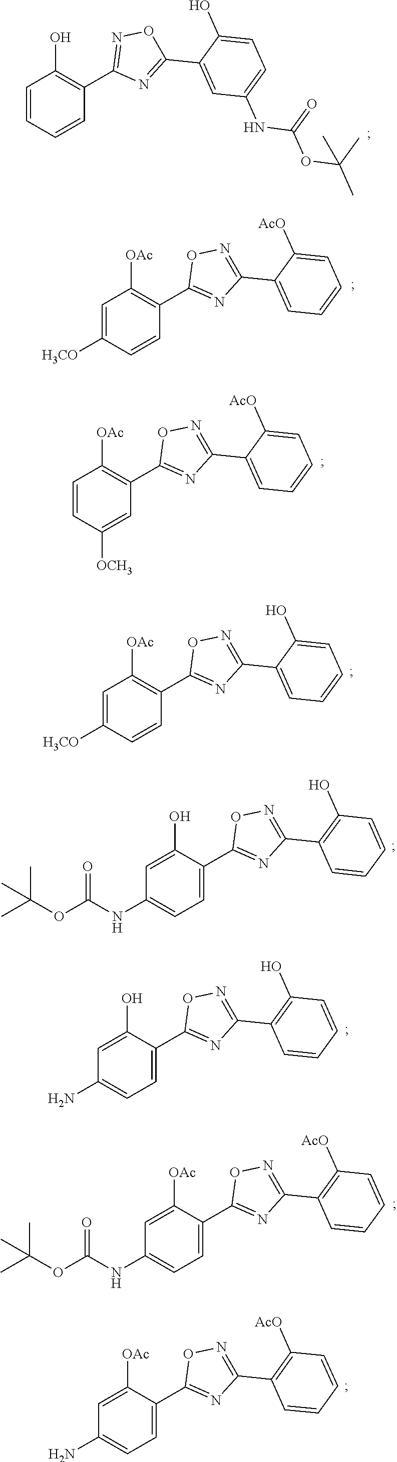

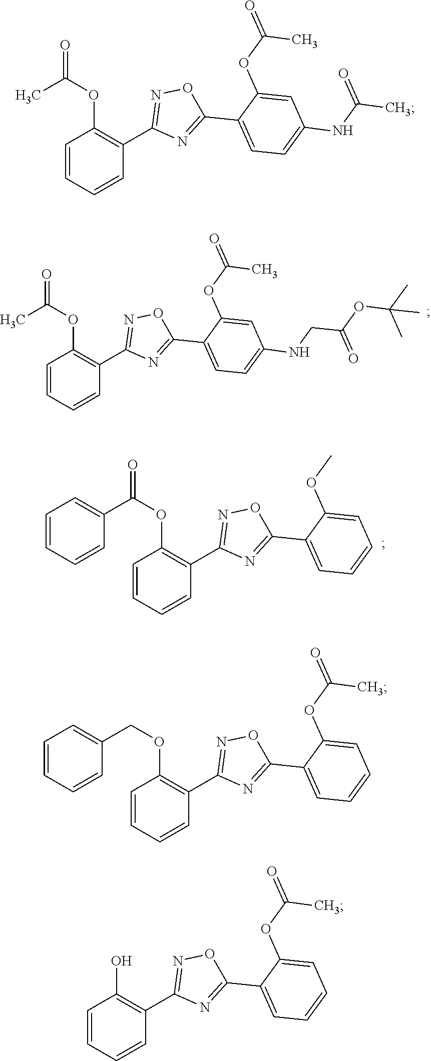 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]

a halogen (e.g., F, Cl, Br) substituted compound thereof; or a pharmaceutically acceptable salt, tautomer, or solvate thereof.

In other embodiments, the RRmod is a thiazole selected from:

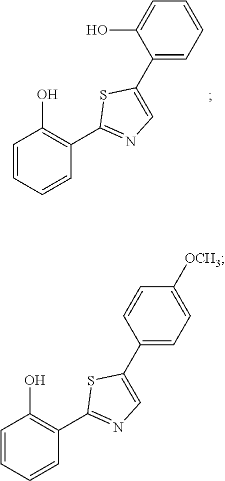 [SMILES: Oc1ccccc1-c1cnc(-c2ccccc2O)s1 ; ";" ; COc1ccc(-c2cnc(-c3ccccc3O)s2)cc1]

a halogen (e.g., F, Cl, Br) substituted compound thereof; or a pharmaceutically acceptable salt, tautomer, or solvate thereof.

In other embodiments, the RRmod is a diazole, triazole, or tetrazole selected from:

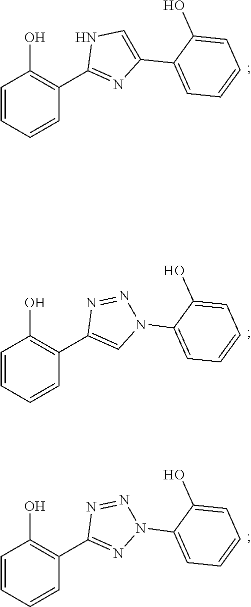 [SMILES: Oc1ccccc1-c1c[nH]c(-c2ccccc2O)n1 ; ";" ; Oc1ccccc1-c1cn(-c2ccccc2O)nn1 ; ";" ; Oc1ccccc1-c1nnn(-c2ccccc2O)n1 ; ";"]

a halogen (e.g., F, Cl, Br) substituted compound thereof; or a pharmaceutically acceptable salt, tautomer, or solvate thereof.

Other embodiments relate to a method of treating a neoplastic disorder. The method includes administering to neoplastic cells of the subject a therapeutically effective amount of a pharmaceutical composition. The pharmaceutical composition includes an RRmod. The therapeutically effective amount of an RRmod is an amount effective to inhibit neoplastic cell growth in the subject.

### BRIEF DESCRIPTION OF THE DRAWINGS

FIGS. 1(A-E) illustrate: (A) the structure of hRRM1 dimer with drug-target sites mapped. The M-site is the hexamer interface, the A-site controls activity, the S-site controls specificity, the C-site is the catalytic site, loop 1 and 2 mediate cross-talk between the S- and C-sites and the P-site binds the smaller R² subunit derived peptide. (B) and (C) illustrate tryptophan fluorescence quenching of hRRM1 in the presence of a phtalimide derivative and a hydrazone (NSAAH) respectively. (D) and (E) show no tryptophan fluorescence quenching of hRRM1 by compounds.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂, alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and Cm alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above.

Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—R_{c} where R_{b} is an alkylene group as defined above and R_{c} is a heterocyclyl radical as defined above. Unless stated otherwise specifically in the specification, a heterocyclylalkyl group can be optionally substituted.

“Heterocyclylalkenyl” refers to a radical of the formula —R_{b}—R_{c} where R_{b} is an alkenylene group as defined above and R_{c} is a heterocyclyl radical as defined above. Unless stated otherwise specifically in the specification, a heterocyclylalkenyl group can be optionally substituted.

“Heterocyclylalkynyl” refers to a radical of the formula —R_{b}—R_{c} where R_{b} is an alkynylene group as defined above and R_{c} is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents. In addition, the aforementioned functional groups may, if a particular group permits, be further substituted with one or more additional functional groups or with one or more hydrocarbyl moieties, such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.

As used herein, the symbol

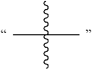

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

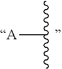

indicates that the chemical entity “A” is bonded to another chemical entity via the point of attachment bond. Furthermore, the specific point of attachment to the non-depicted chemical entity can be specified by inference. For example, the compound

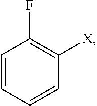

wherein X is

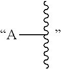

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

The term “neoplasm” refers to any abnormal mass of cells or tissue as a result of neoplasia. The neoplasm may be benign, potentially malignant (precancerous), or malignant (cancerous). An adenoma is an example of a neoplasm.

The terms “adenoma”, “colon adenoma” and “polyp” are used herein to describe any precancerous neoplasm of the colon.

The term “colon” as used herein is intended to encompass the right colon (including the cecum), the transverse colon, the left colon and the rectum.

The terms “colorectal cancer” and “colon cancer” are used interchangeably herein to refer to any cancerous neoplasia of the colon (including the rectum, as defined above).

The terms “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The terms “healthy” and “normal” are used interchangeably herein to refer to a subject or particular cell or tissue that is devoid (at least to the limit of detection) of a disease condition.

The term “nucleic acid” refers to polynucleotides such as deoxyribonucleic acid (DNA), and, where appropriate, ribonucleic acid (RNA). The term should also be understood to include analogues of either RNA or DNA made from nucleotide analogues, and, as applicable to the embodiment being described, single-stranded (such as sense or antisense) and double-stranded polynucleotides. In some embodiments, “nucleic acid” refers to inhibitory nucleic acids. Some categories of inhibitory nucleic acid compounds include antisense nucleic acids, RNAi constructs, and catalytic nucleic acid constructs. Such categories of nucleic acids are well-known in the art.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The term “anticancer agent” refers to a compound which treats a cancer (e.g., a compound which is useful in the treatment of a cancer). The anticancer effect(s) may arise through one or more mechanisms including, but not limited to, the regulation of cell proliferation, the inhibition of cell cycle progression, the inhibition of cell growth, the inhibition of angiogenesis, the inhibition of metastasis, the inhibition of invasion (e.g., the spread of tumor cells into healthy neighboring tissue), or the promotion of apoptosis. The term “antineoplastic” is used herein to mean a chemotherapeutic intended to inhibit or prevent the maturation and proliferation of neoplasms, by targeting the DNA.

The term “cell growth” is used in the contexts of cell development and cell division (reproduction). When used in the context of cell division, it refers to growth of cell populations, where one cell (the “mother cell”) grows and divides to produce two “daughter cells” (M phase). When used in the context of cell development, the term refers to increase in cytoplasmic and organelle volume (G1 phase), as well as increase in genetic material before replication (G2 phase).

The terms “neoplastic cell”, “cancer cell” or “tumor cell” refer to cells that divide at an abnormal (i.e., increased) rate. A neoplastic cell or neoplasm (tumor) can be benign, potentially malignant, or malignant. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, meningoma, medulloblastoma, schwannoma and epidymoma.

The terms “treating” or “treatment” of a condition may refer to preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition’ or some combination thereof. With regard to neoplastic disorders, “treating” or “treatment” may refer to inhibiting or slowing neoplastic and/or malignant cell growth, proliferation, and/or metastasis, preventing or delaying the development of neoplastic and/or malignant cell growth, proliferation, and/or metastasis, or some combination thereof. With regard to a tumor, “treating” or “treatment” may refer to eradicating all or part of a tumor, inhibiting or slowing tumor growth and metastasis, preventing or delaying the development of a tumor, or some combination thereof.

The phrase “therapeutically effective amount” refers to an amount of a compound that produces a desired therapeutic effect. In one aspect, the therapeutically effective amount is the amount required to inhibit neoplastic cell growth in the subject. The precise therapeutically effective amount is an amount of the composition that will yield the most effective results in terms of efficacy in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy (Gennaro ed. 22nd Edition, Pharmaceutical Press, London, UK, 2012).

The term “epitope” refers to a physical structure on a molecule that interacts with a selective component, e.g., the selective component such as an RRmod described herein. In exemplary embodiments, epitope refers to a desired region on a target molecule that specifically interacts with a selectivity component.

Embodiments described herein relate to ribonucleotide reductase modulators (RRmods), pharmaceutical compositions comprising RRmods, therapeutic uses of RRmods, as well as compounds found to be specifically effective as allosteric modulators of ribonucleotide reductase activity in neoplastic cells.

Ribonucleotide reductase enzyme activity is required for de novo DNA synthesis by catalyzing ribonucleotides to deoxy ribonucleotides and maintaining a balanced nucleotide precursor molecule pool. Since the proliferation of cancer cells requires excess dNTPs for DNA synthesis, it is believed that RRmods that specifically target RR1 can be employed to inhibit cell growth and proliferation of neoplastic cells through the modulation of ribonucleotide reductase enzyme activity.

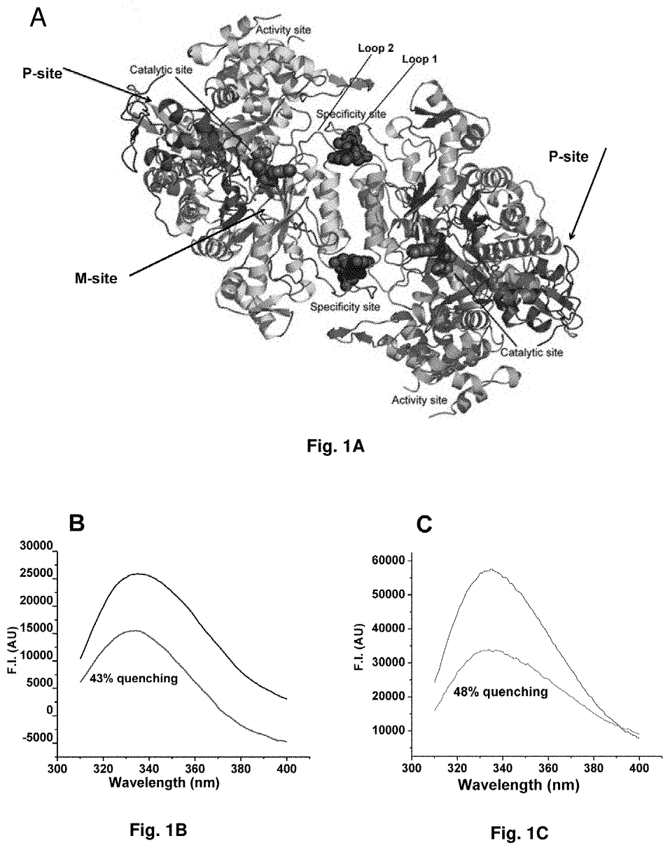

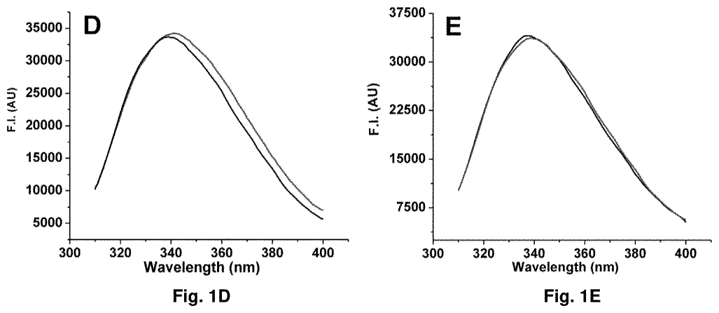

It was found that the large subunit (α-subunit or hRRM1) of ribonucleotide reductase (RR) includes four potentially druggable sites (see FIG. 1A). These sites include the A (activity)-site, the S (specificity)-site, the C (catalytic)-site and the P (peptide)-site. Using X-ray crystallography, an additional epitope of hRRM1, the M-site, was found to be in the hexamer interface of hRRM1. The M-site is a surface pocket including residues constituting the (3-cap located on one dimer and the loop involving residue 480 belonging to an adjacent dimer at the hexamer interface.

It was found that the M-site can be targeted by small molecules to modulate ribonucleotide reductase activity. Using in silico high throughput screening and RR activity and growth inhibition cell culture in vitro assays, small molecules that bind to or complex with M-site or the catalytic C-site of hRRM1 were identified that were capable of allosterically inhibiting or activating the enzyme. These identified small molecules and analogs thereof can be used in a method of modulating ribonucleotide reductase activity in a neoplastic cell to inhibit neoplastic cell growth.

In some embodiments, RRmods described herein include agents capable of binding to or complexing with an epitope of hRRM1. In some embodiments the RRmod binds to the hexamer interface M-site or the catalytic C-site of hRRM1, and allosterically modulates ribonucleotide reductase enzyme activity, thereby affecting de novo DNA synthesis, cell growth and proliferation of neoplastic cells.

In certain embodiments, the RRmod is a small molecule. Exemplary data of small molecule compounds found to be specifically effective as allosteric modulators of ribonucleotide reductase activity are provided in the Examples below. In particular, the disclosed compounds had activity in inhibiting the ribonucleotide reductase activity in DNA synthesis assays and for killing carcinomas in a cell-based assay, generally with a micromolar IC₅₀.

In some embodiments, the RRmod can include a compound having the following formula (I):

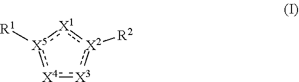

or a pharmaceutically acceptable salt, tautomer, or solvate thereof;

wherein:

X¹, X³, and X⁴ are each independently C, S, O, or N;

X² and X⁵ are each independently C or N;

at least two but no more than four of X¹, X², X³, X⁴, or X⁵ is not C;

---- is an optional bond;

R¹ and R² are each independently an alkyl, aryl, heteroaryl, or heterocyclyl optionally substituted with one or more R⁴; and

each R⁴ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety, and wherein R¹ and R² are not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define an oxadiazole, thiazole, diazole, triazole, or tetrazole.

In some embodiments, each R⁴ is independently halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R¹ and R² are not benzoic acid.

In other embodiments, R¹ and R² are each independently selected from the group consisting of:

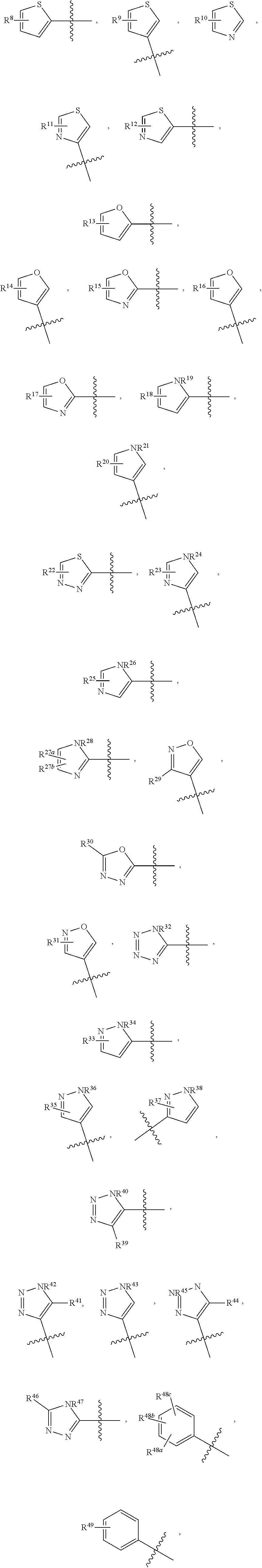

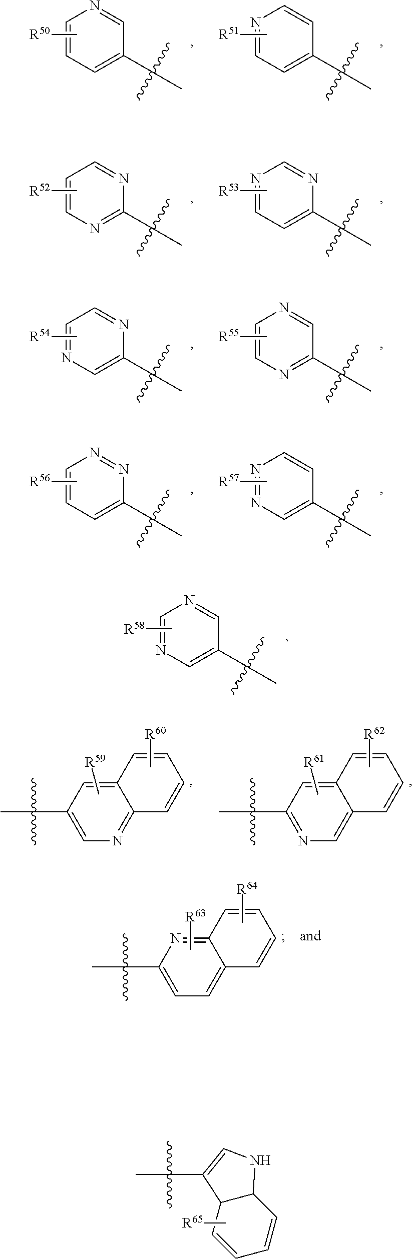

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R¹ and R² are each independently a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

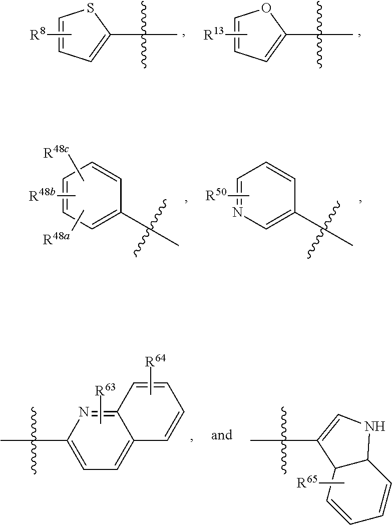

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In still other embodiments, the compound or RRmod can have a molecular weight (g/mol) less than about 400 g/ml, less than about 390 g/mol, less than about 380 g/mol, less than about 370 g/mol less than about 360 g/mol, less than about 350 g/mol, less than about 340 g/mol, less than about 330 g/mol, less than about 320 g/mol, less than about 310 g/mol, less than about 300 g/mol, less than about 290 g/mol, less than about 280 g/mol, less than about 270 g/mol, or less than about 260 g/mol. For example, the compound can have molecular weight (g/mol) of about 230 g/mol to about 400 g/mol, about 240 g/mol to about 360 g/mol or about 250 g/mol to about 350 g/mol.

In some embodiments, the compound or RRmod can inhibit the enzymatic activity of recombinant human ribonucleotide reductase at an IC₅₀ of less than 10 at an IC₅₀ of less than 5 μM, at an IC₅₀ of less than 1 μM, at an IC₅₀ of less than 250 nM, at an IC₅₀ of less than 50 nM, at an IC₅₀ of less than 10 nM, at an IC₅₀ of less than 5 nM at a recombinant, at an IC₅₀ of about 2.5 nM to about 10 nM, or less than about 2.5 nM.

In some embodiments, the RRmod can include a compound having the following formula (II):

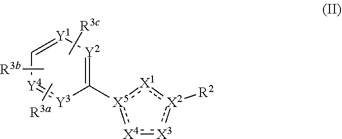

or a pharmaceutically acceptable salt, tautomer, or solvate thereof;

wherein:

X¹, X³, and X⁴ are each independently C, S, O, or N;

X² and X⁵ are each independently C or N;

at least two but no more than four of X¹, X², X³, X⁴, or X⁵ is not C;

Y¹, Y², Y³, Y⁴ are each independently N or C, provided no more than 3 of Y¹, Y², Y³, or Y⁴ are N;

---- is an optional bond;

R² is an alkyl, aryl, heteroaryl, or heterocyclyl optionally substituted with one or more R⁴;

R^{3a}, R^{3b}, R^{3c}, and each R⁴ are each independently absent, halogen, or optionally substituted hydroxyl, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, heterocyclyl, alkylaryl, aromatic moiety, or heteroaromatic moiety, and R² is not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define an oxadiazole, thiazole, diazole, triazole, or tetrazole.

In some embodiments, R^{3a}, R^{3b}, and R^{4c} are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R² is not benzoic acid.

In some embodiments, R² is independently selected from the group consisting of:

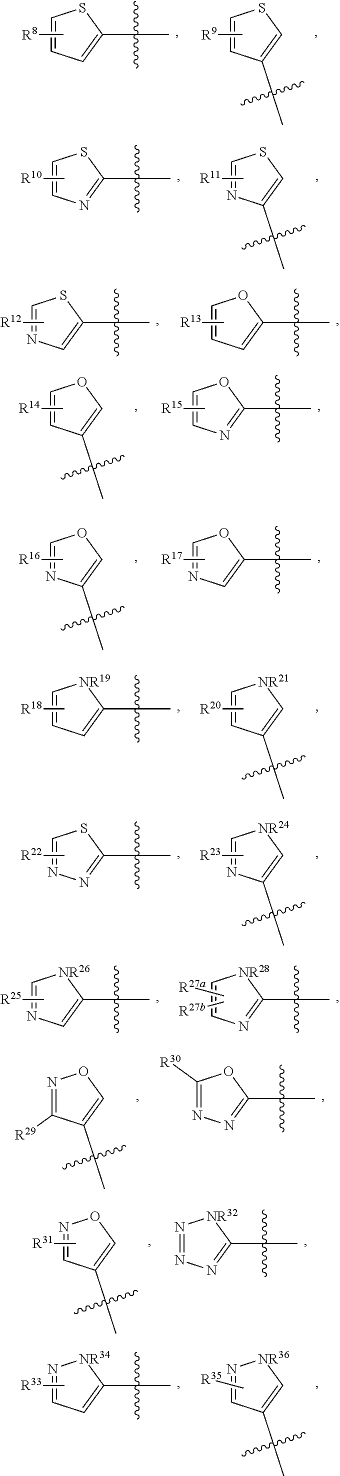

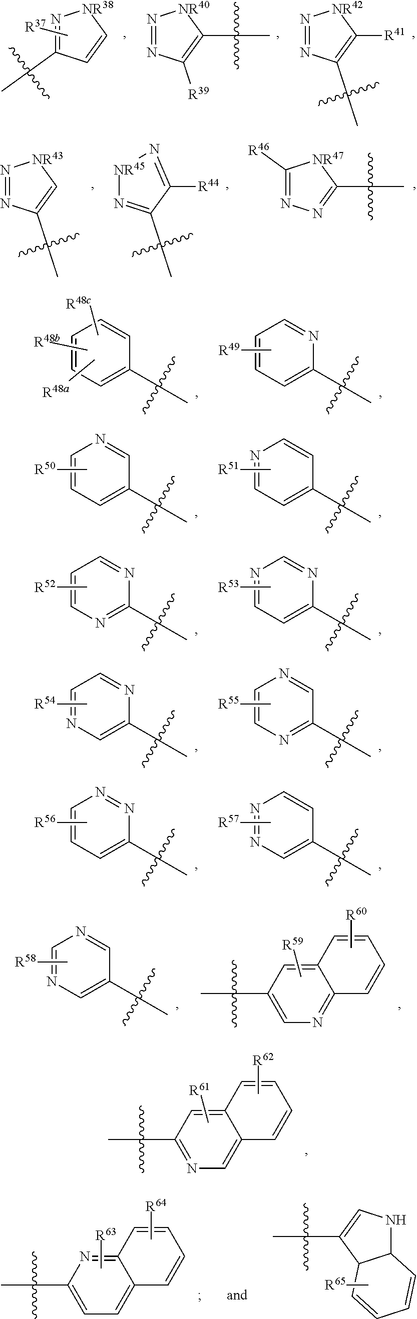

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

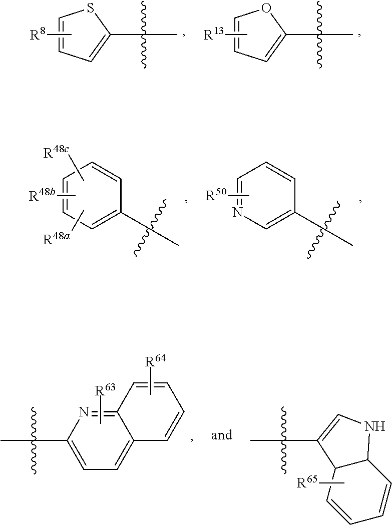

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, the RRmod can include a compound selected from:

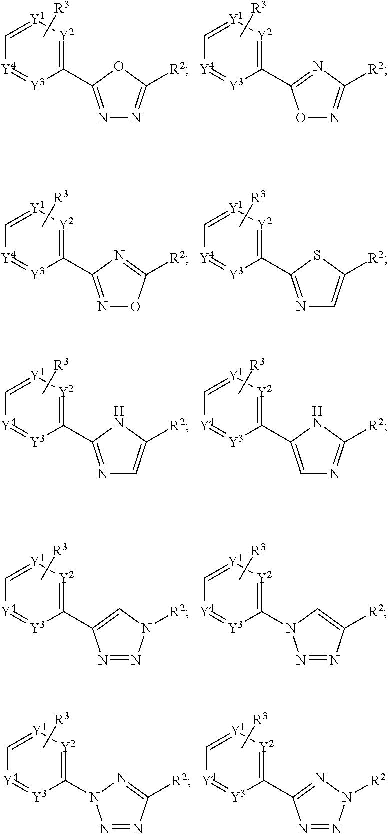

or a pharmaceutically acceptable salt, tautomer, or solvate thereof

wherein;

Y¹, Y², Y³, Y⁴ are each independently N or C, provided no more than 3 of Y¹, Y², Y³, or Y⁴ are N;

R² is an alkyl, aryl, heteroaryl, or heterocyclyl optionally substituted with one or more R⁴;

R³ and R⁴ are each independently absent, halogen, or optionally substituted hydroxyl, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, heterocyclyl, alkylaryl, aromatic moiety, or heteroaromatic moiety, and R² is not a naphtholyl.

In some embodiments, each R³ is independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R² is not benzoic acid.

In some embodiments, R² is independently selected from the group consisting of:

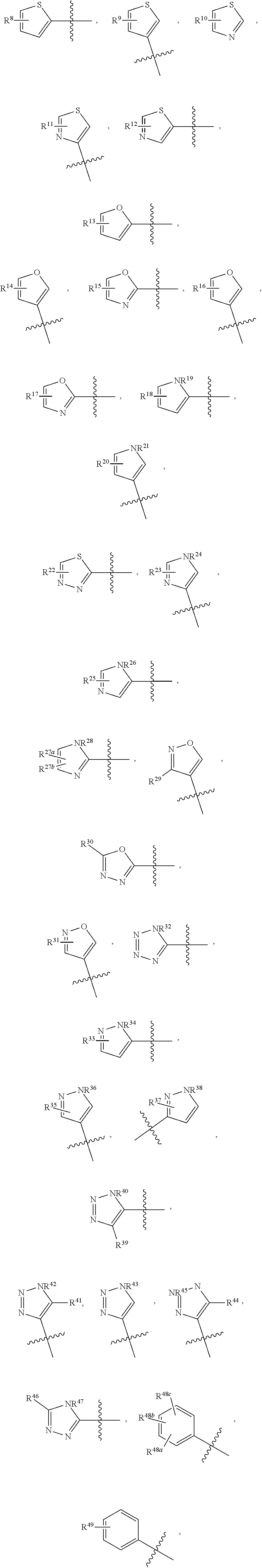

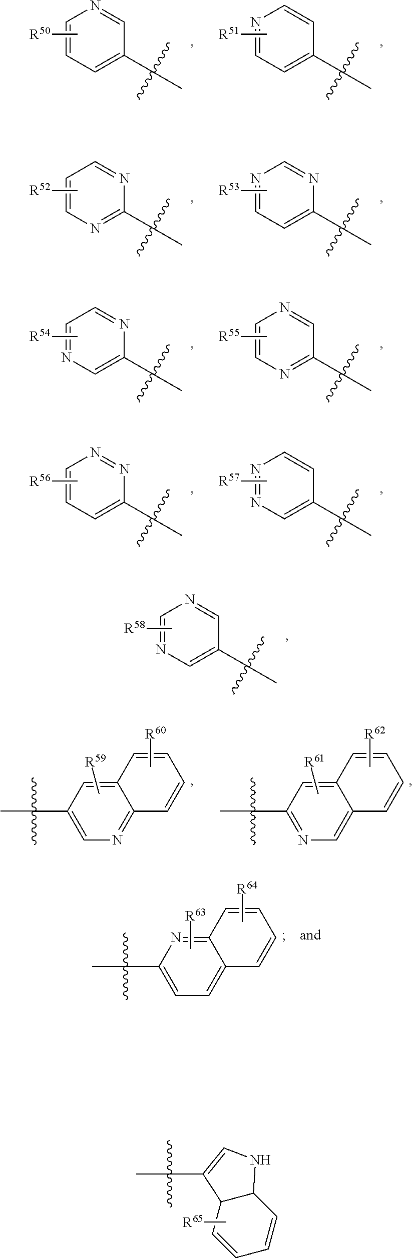

R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R₄₅, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

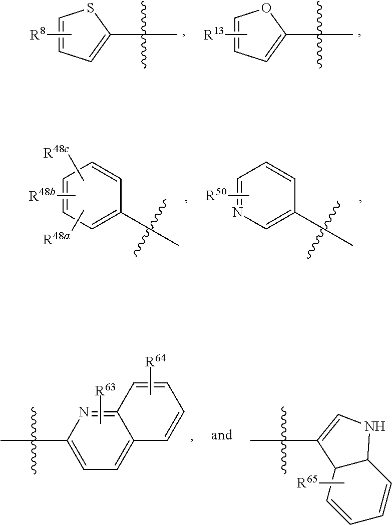

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, the RRmod can include a compound having the following formula (III):

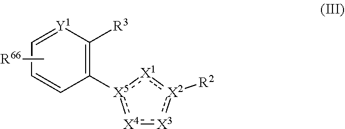

or a pharmaceutically acceptable salt, tautomer, or solvate thereof, wherein:

wherein:

X¹, X³, and X⁴ are each independently C, S, O, or N;

X² and X⁵ are each independently C or N;

at least two but no more than four of X¹, X², X³, X⁴, or X⁵ is not C;

Y¹ is Nor C;

---- is an optional bond;

R² is an alkyl, aryl, heteroaryl, or heterocyclyl optionally substituted with one or more R⁴;

R³ and R⁴ are each independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;

R⁶⁶ is absent, halogen, alkoxy, or amino; and

R² is not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define oxadiazole, thiazole, diazole, triazole, or tetrazole.

In some embodiments, R³ is halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R² is not benzoic acid.

In some embodiments, R² is independently selected from the group consisting of:

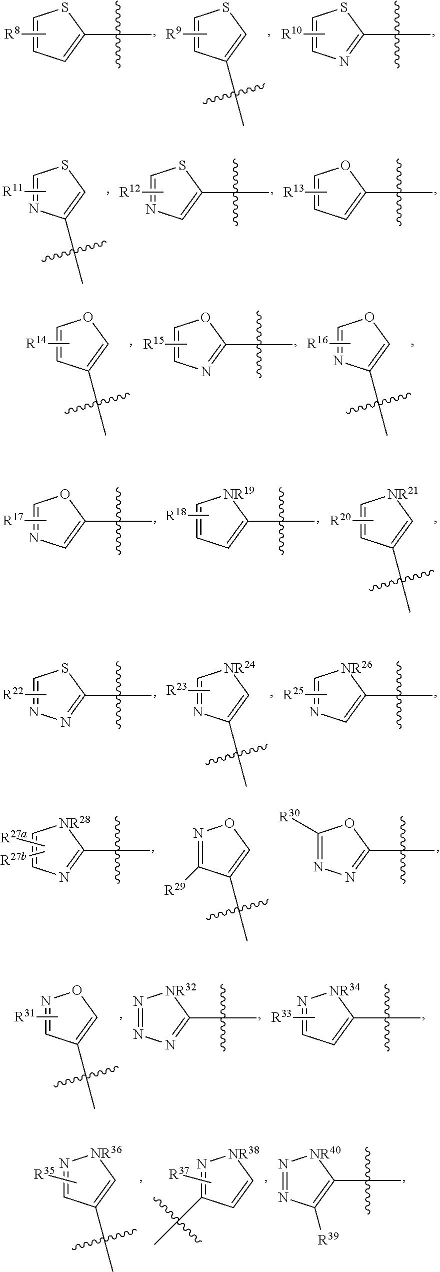

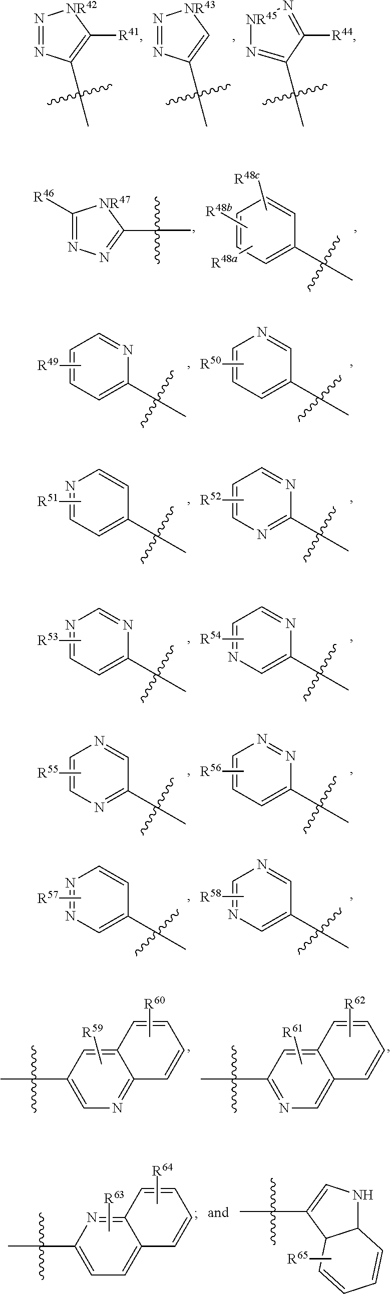

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

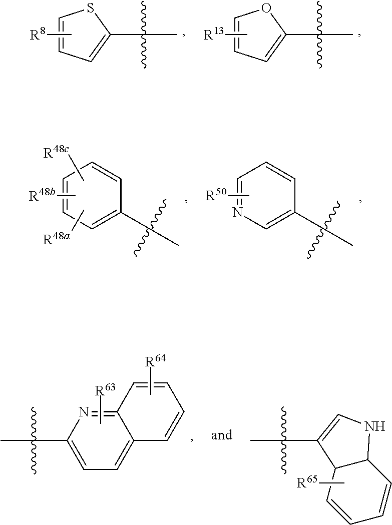

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, the RRmod can include a compound selected from:

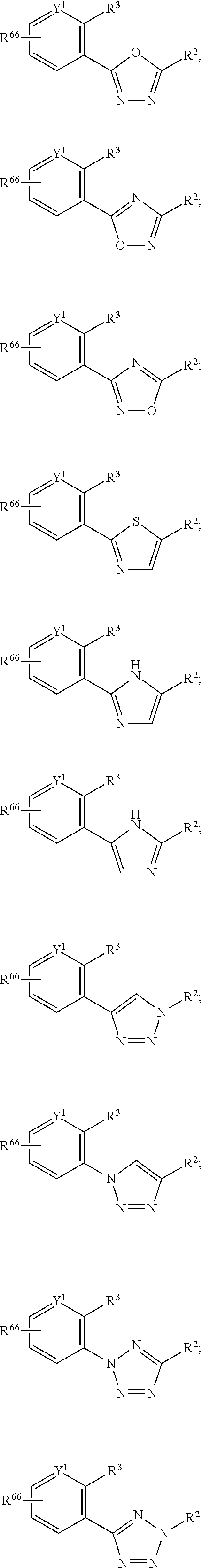

or a pharmaceutically acceptable salt, tautomer, or solvate thereof

wherein;

Y¹ is Nor C;

R² is an alkyl, aryl, heteroaryl, or heterocyclyl optionally substituted with one or more R⁴;

R³ and R⁴ are each independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;

R⁶⁶ is absent, halogen, alkoxy, or amino; and

R² is not a naphtholyl.

In some embodiments, R³ is halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, R² is not benzoic acid.

In some embodiments, R² is independently selected from the group consisting of:

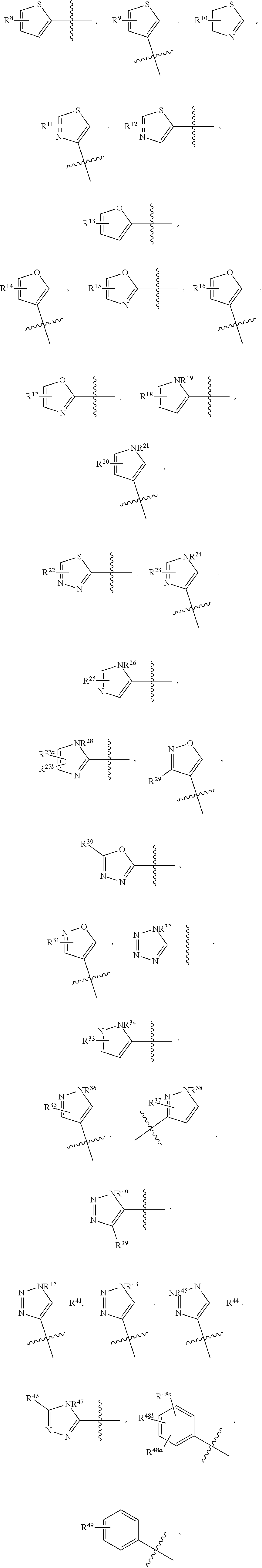

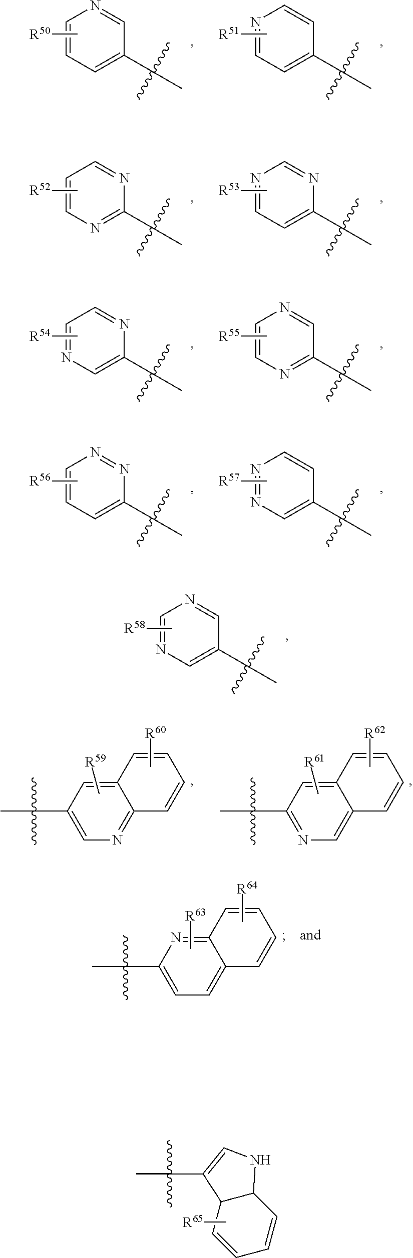

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

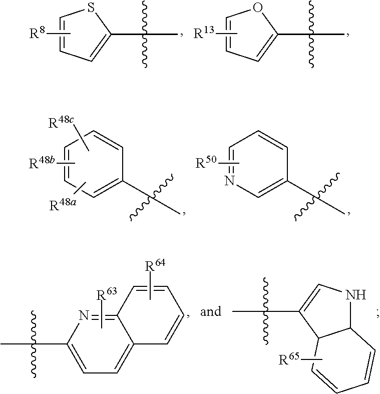

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, the RRmod can include a compound having the following formula (IV):

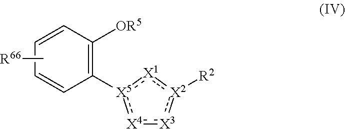

or a pharmaceutically acceptable salt, tautomer, or solvate thereof, wherein:

wherein:

X¹, X³, and X⁴ are each independently C, S, O, or N;

X² and X⁵ are each independently C or N;

at least two but no more than four of X¹, X², X³, X⁴, or X⁵ is not C;

---- is an optional bond;

R² is an alkyl, aryl, heteroaryl, or heterocyclyl optionally substituted with one or more R⁴;

each R⁴ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;

R⁵ is H, C₁-C₆ alkyl, aryl, or —(CO)-alkyl;

R⁶⁶ is absent, halogen, alkoxy, or amino; and

R² is not a naphtholyl.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define and oxadiazole, thiazole, diazole, triazole, or tetrazole.

In some embodiments, R² is not benzoic acid.

In some embodiments, R² is independently selected from the group consisting of:

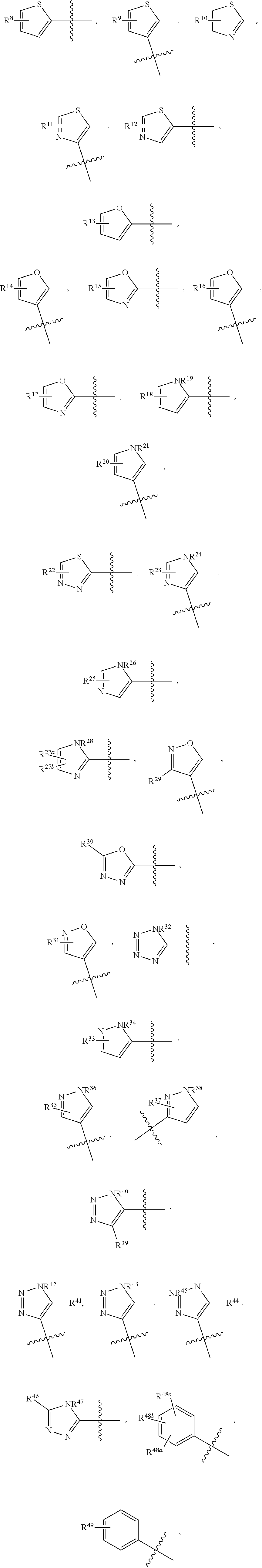

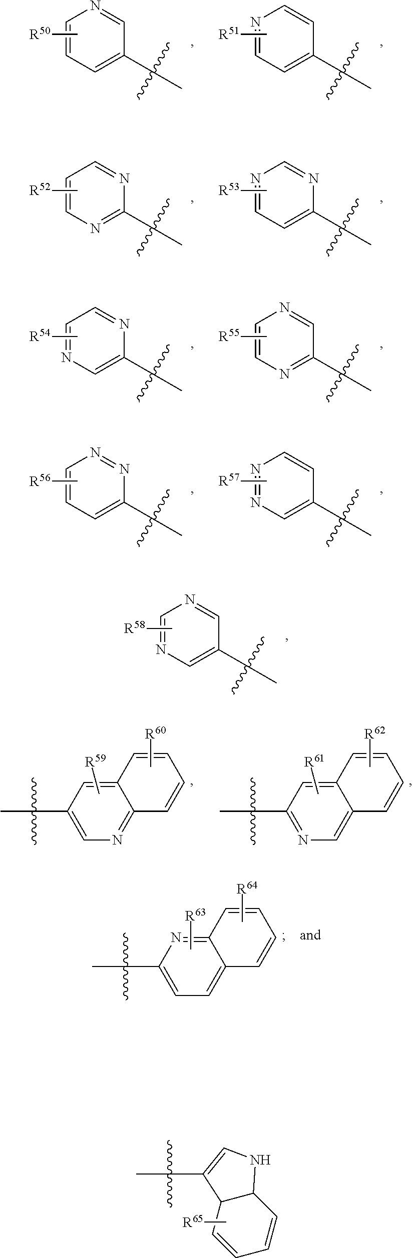

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

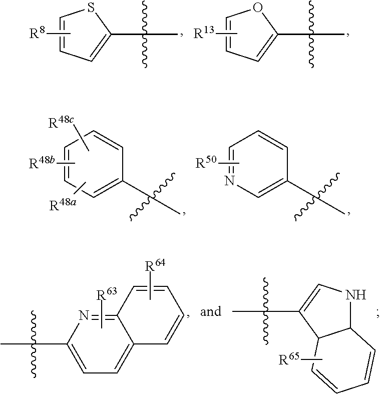

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, the RRmod can include a compound selected from:

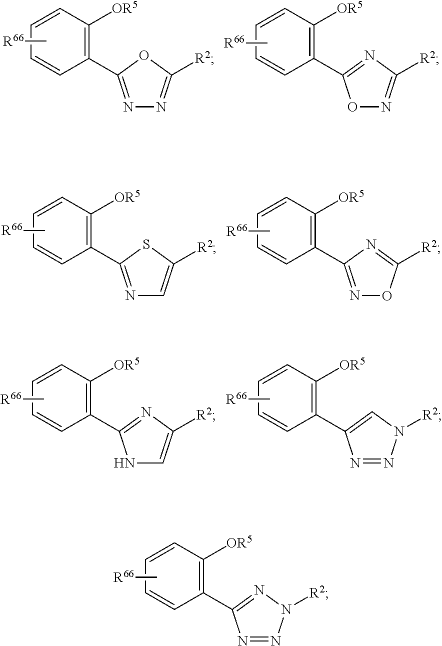

or a pharmaceutically acceptable salt, tautomer, or solvate thereof:

wherein;

R² is an alkyl, aryl, heteroaryl, or heterocyclyl optionally substituted with one or more R⁴;

R² is an alkyl, aryl, heteroaryl, or heterocyclyl optionally substituted with one or more R⁴;

each R⁴ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;

R⁵ is H, C₁-C₆ alkyl, aryl, or —(CO)-alkyl;

R⁶⁶ is absent, halogen, alkoxy, or amino;

and R² is not a naphtholyl.

In some embodiments, R² is not benzoic acid.

In some embodiments, R² is independently selected from the group consisting of:

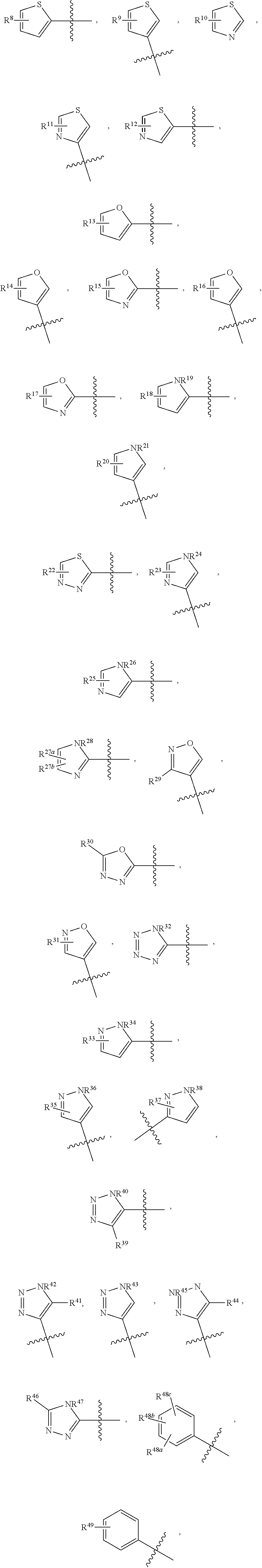

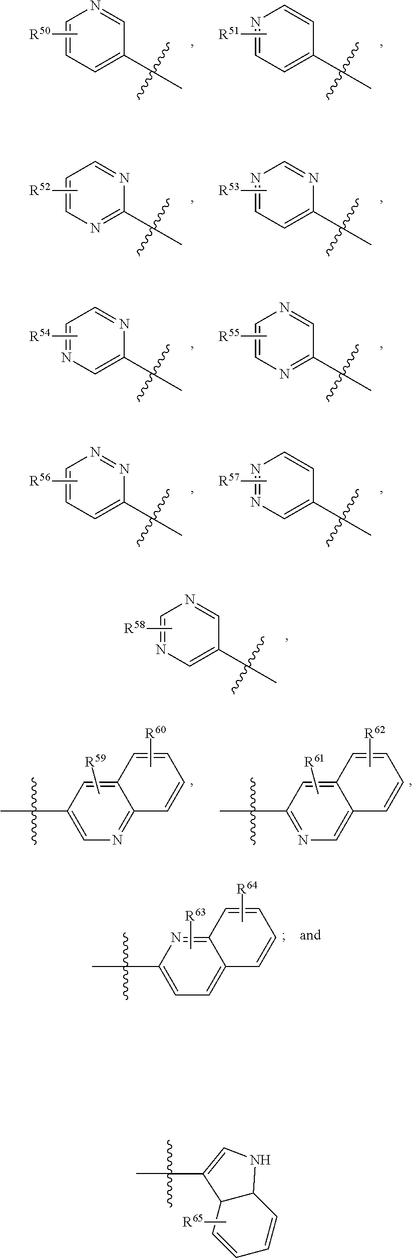

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, R² is selected from the group consisting of:

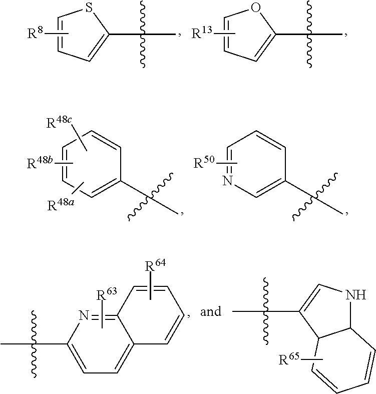

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, a compound having formula (IV) can include the following:

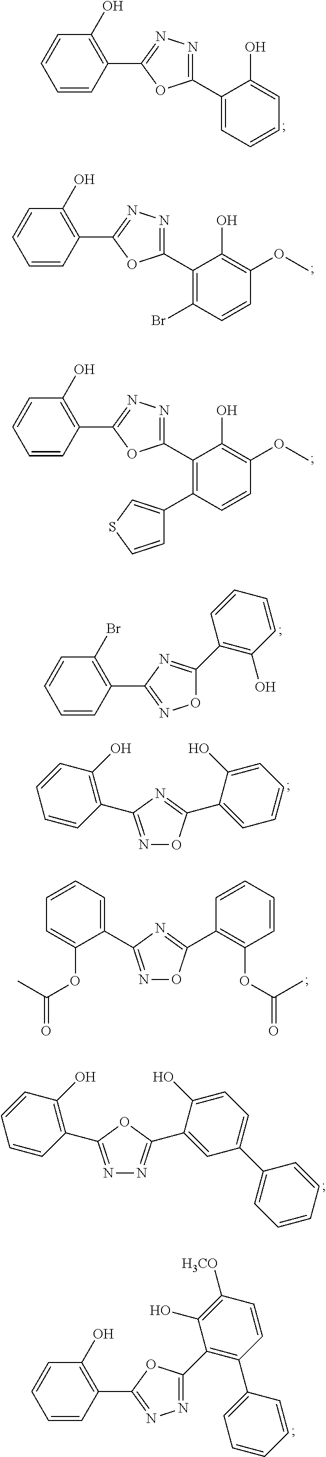

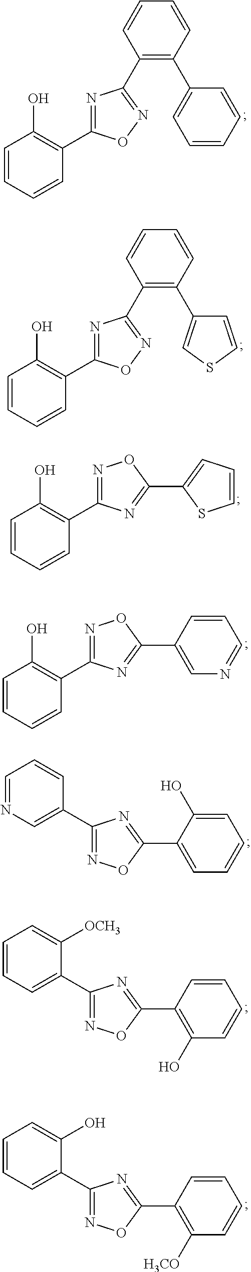

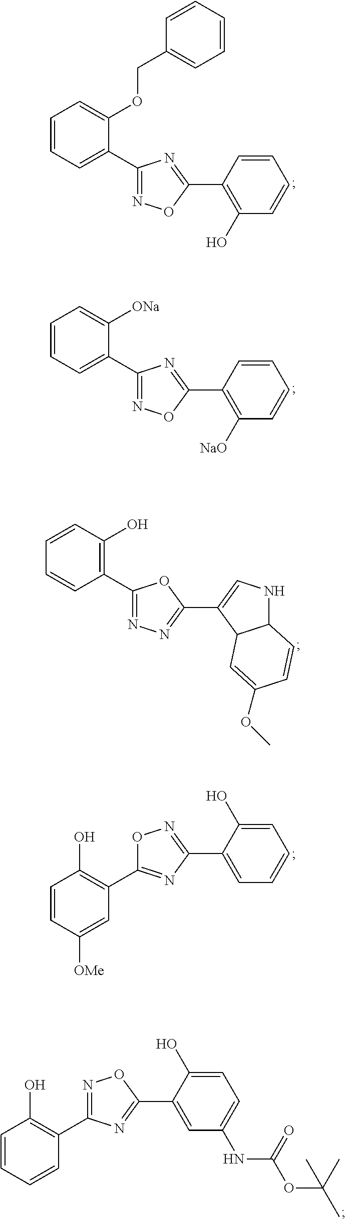

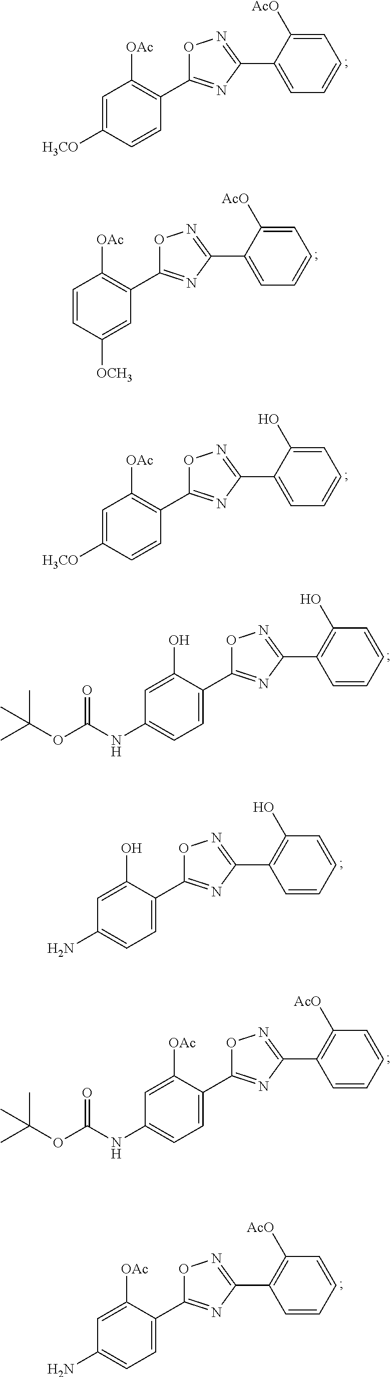

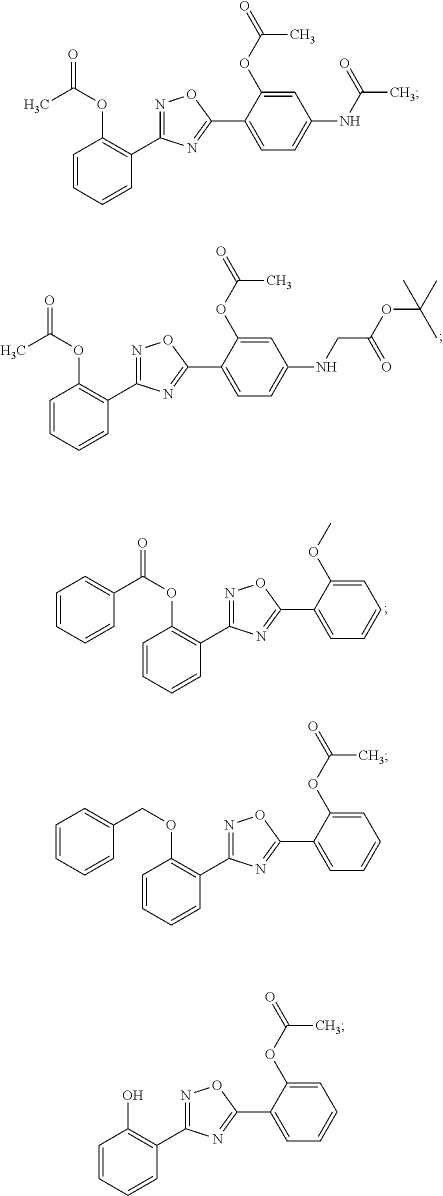 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]

a halogen (e.g., F, Cl, Br) substituted compound thereof; or a pharmaceutically acceptable salt, tautomer, or solvate thereof.

In some embodiments, the RRmod can include a compound having the following formula (V):

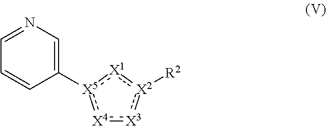

or a pharmaceutically acceptable salt, tautomer, or solvate thereof, wherein:

X¹, X², and X³ are each independently N or O, and two of X¹, X², and X³ are N;

---- is an optional bond;

R² is an aryl group or heteroaryl group optionally substituted with one or more R³; and

each R³ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety, and wherein R² is not a naphthol group.

In some embodiments, at least one of X¹, X³, or X⁴ is N. For example, one, two, or three of X¹, X³, or X⁴ is N.

In other embodiments, at least one of X² or X⁵ is C.

In some embodiments, X¹, X², X³, X⁴, and X⁵ define an oxadiazole, thiazole, diazole, triazole, or tetrazole.

In some embodiments, R² is not benzoic acid.

In some embodiments, R² is independently selected from the group consisting of:

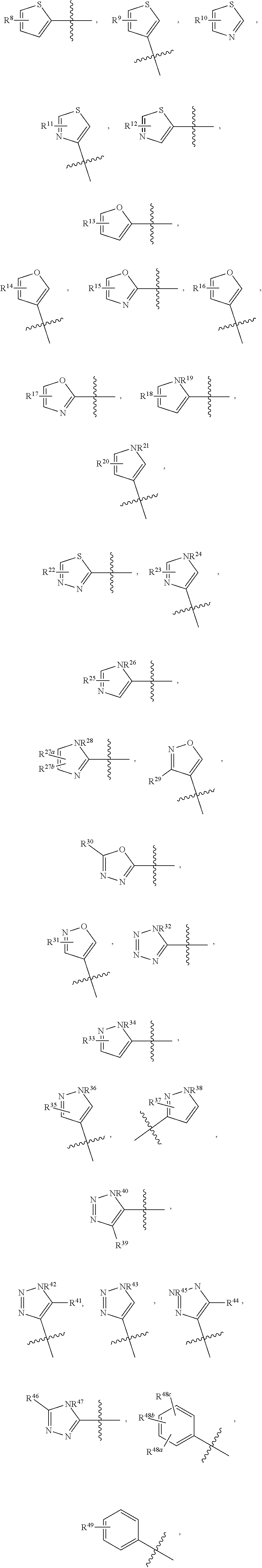

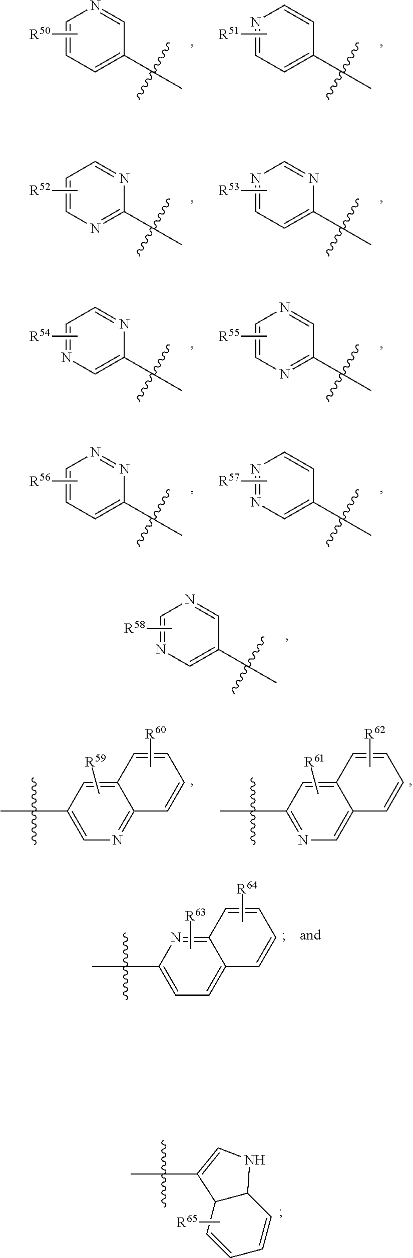

wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl that is optionally substituted with one or more halogen, hydroxyl, alkyl, alkoxy, alkylcarbonyl, aryl, heteroaryl, heterocyclyl, or a pharmaceutically acceptable salt thereof.

In other embodiments, R² is selected from the group consisting of:

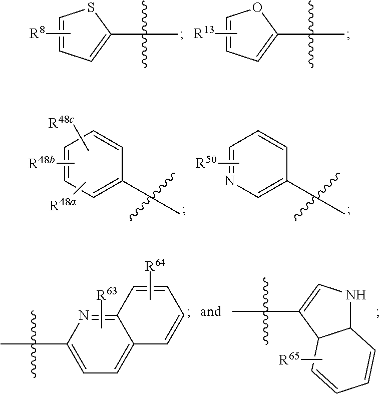

wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In other embodiments, the RRmod can include a compound selected from:

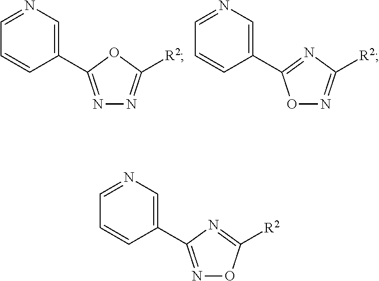

or a pharmaceutically acceptable salt, tautomer, or solvate thereof, wherein:

R² is an aryl group or heteroaryl group optionally substituted with one or more R³; and each R³ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety, and wherein R² is not a naphthol group.

In some embodiments, R² is not benzoic acid.

In some embodiments, R² is independently selected from the group consisting of:

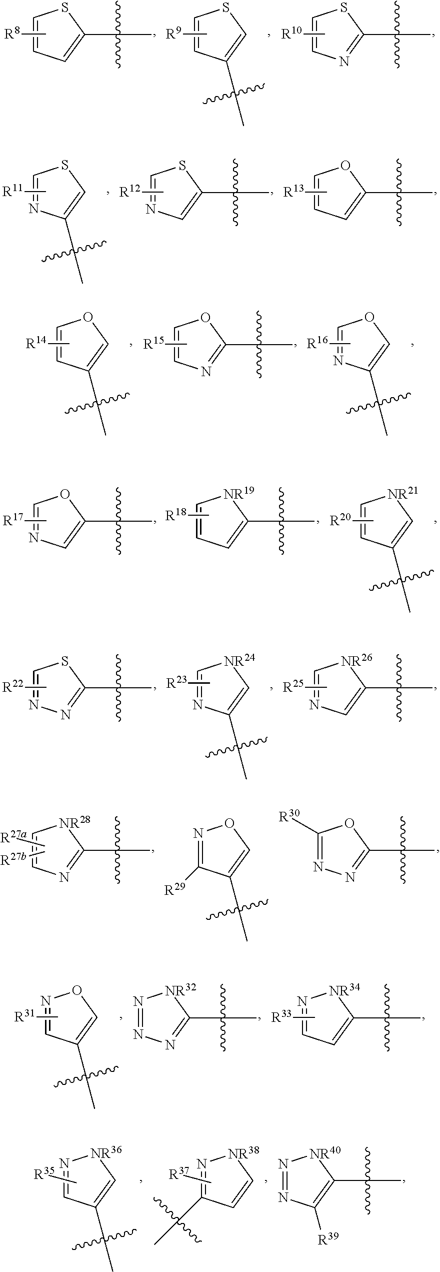

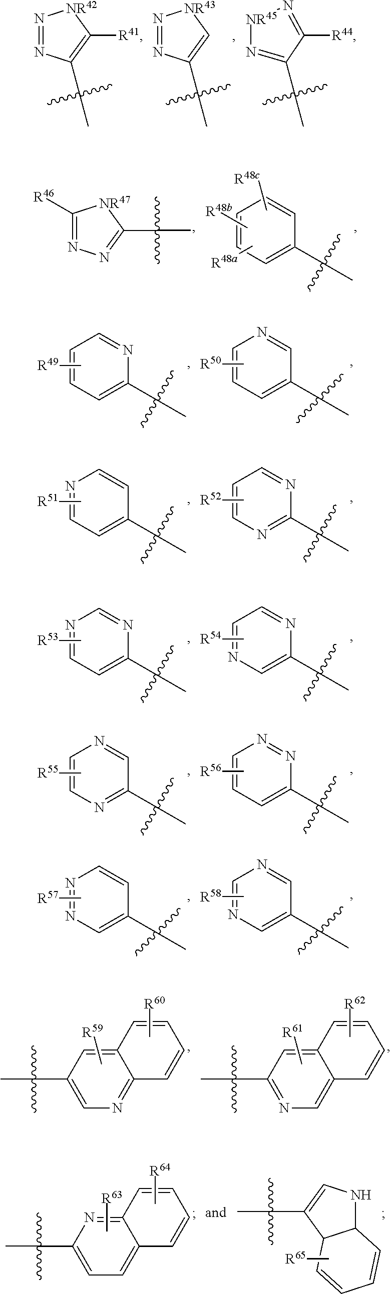

wherein R⁸, R⁹, R¹⁰, R¹¹, R¹², R¹³, R¹⁴, R¹⁵, R¹⁶, R¹⁷, R′8, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, R² is a thiophenyl, furanyl, phenyl, pyridinyl, pyridazinyl, pyrimidinyl, triazinyl, quinolinyl, or indolyl that is optionally substituted with one or more halogen, hydroxyl, alkyl, alkoxy, alkylcarbonyl, aryl, heteroaryl, heterocyclyl, or a pharmaceutically acceptable salt thereof.

In other embodiments, R² is selected from the group consisting of:

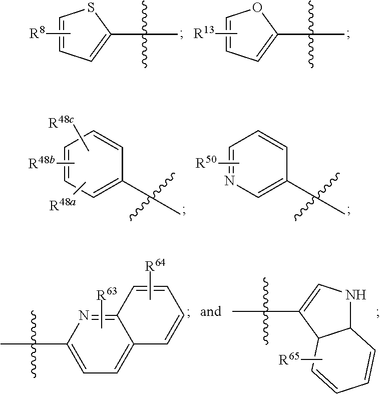

wherein R⁸, R¹³, R^{48a}, R^{48b}, R^{48c}, R⁵⁰, R⁶³, R⁶⁴, R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

In some embodiments, a compound having formula (V) can include the following compounds:

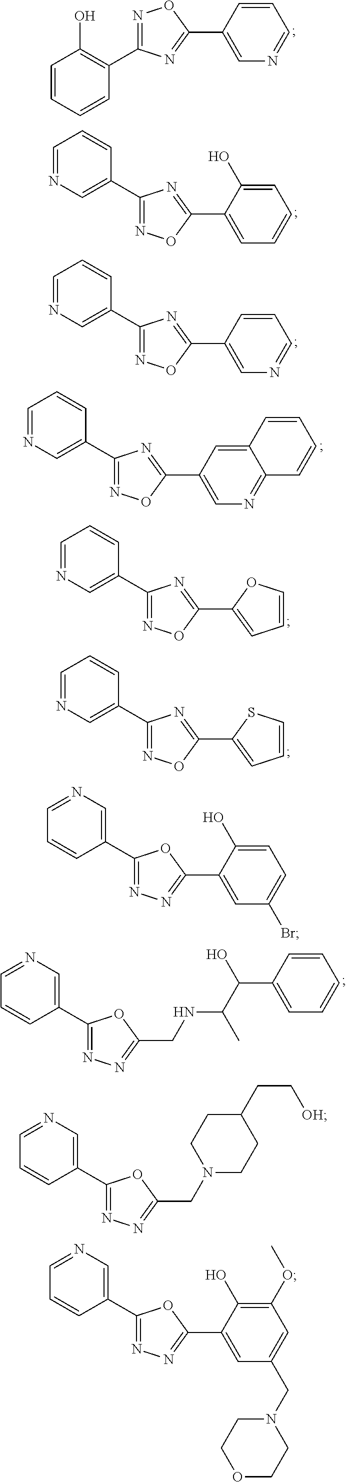

a halogen (e.g., F, Cl, Br) substituted compound thereof; or a pharmaceutically acceptable salt, tautomer, or solvate thereof.

In other embodiments, the RRmod can be a compound selected from:

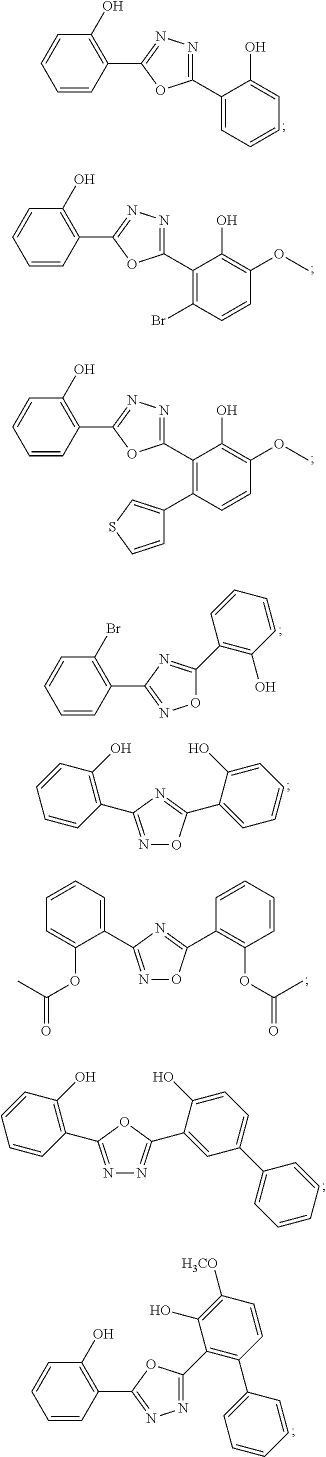

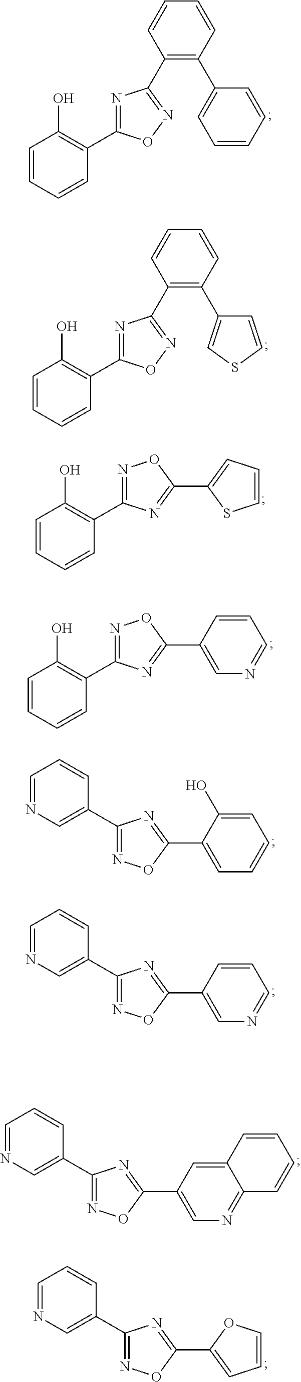

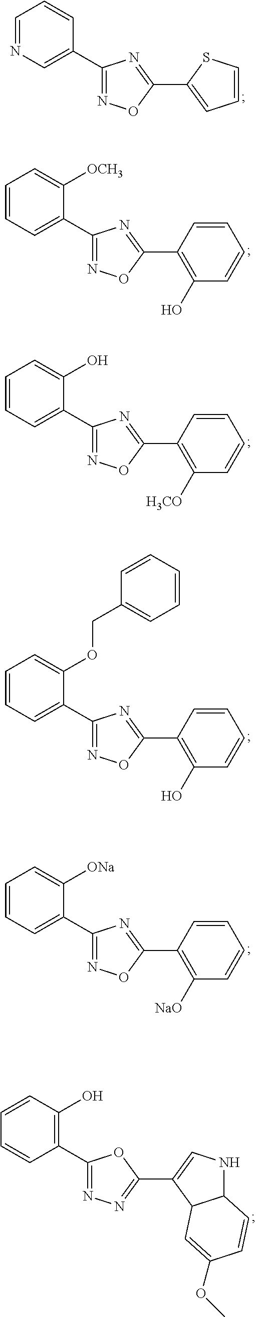

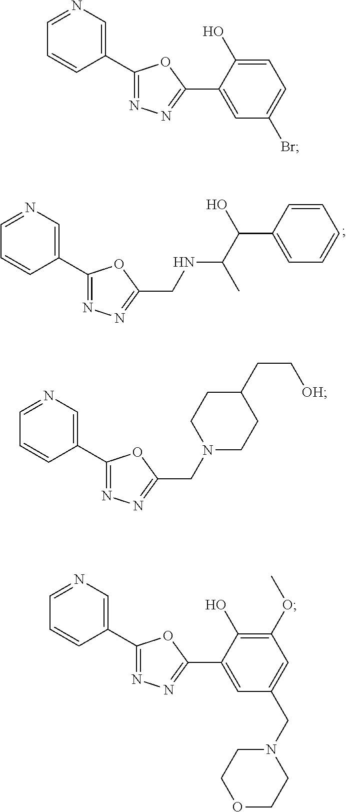 [SMILES: Oc1ccc(Br)cc1-c1nnc(-c2cccnc2)o1 ; CC(NCc1nnc(-c2cccnc2)o1)C(O)c1ccccc1 ; OCCC1CCN(Cc2nnc(-c3cccnc3)o2)CC1 ; COc1cc(CN2CCOCC2)cc(-c2nnc(-c3cccnc3)o2)c1O]

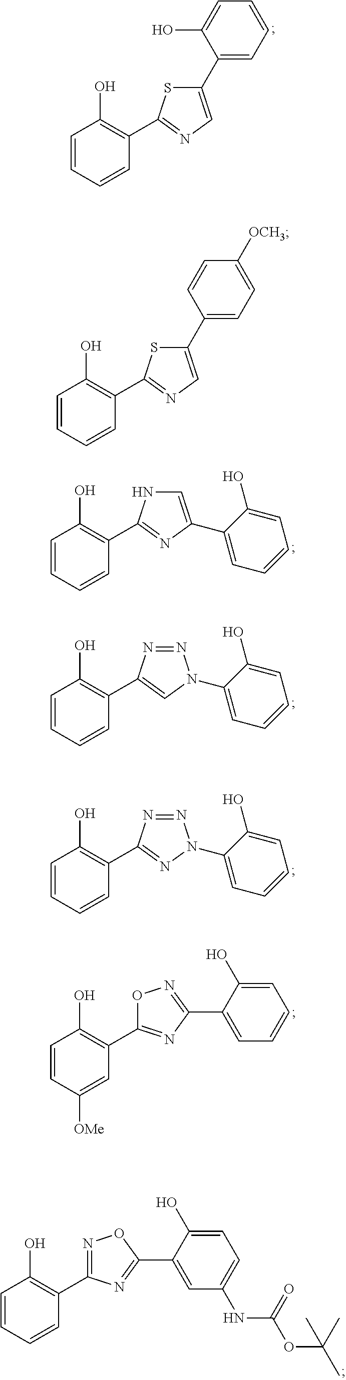

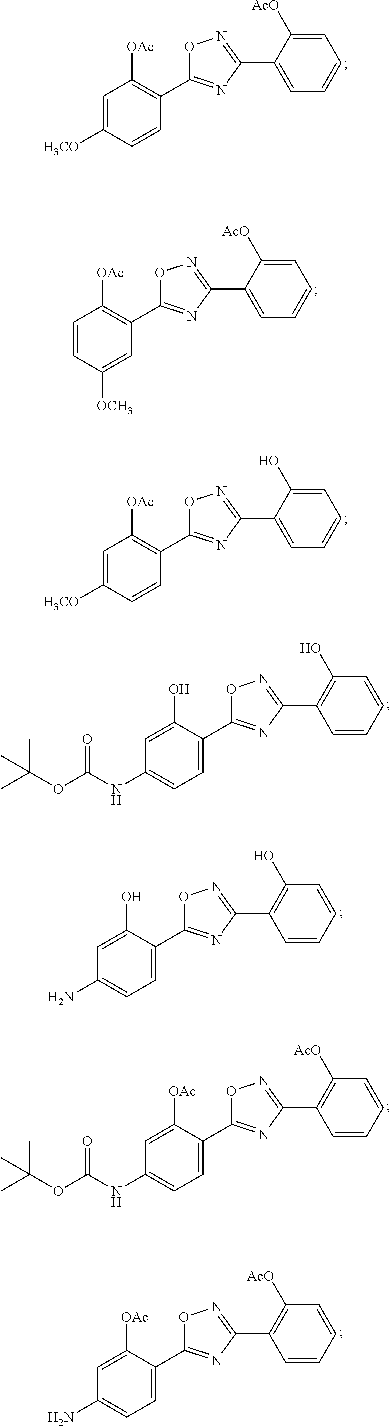

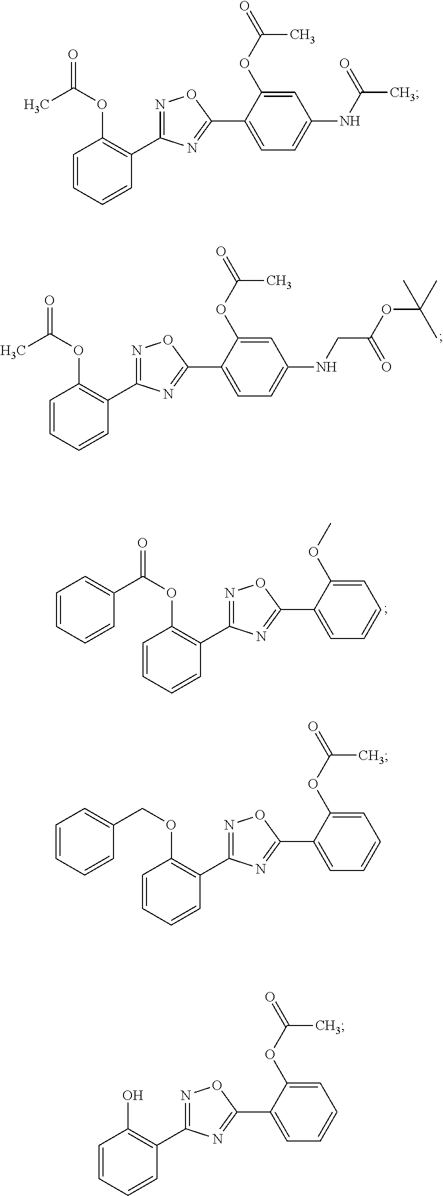 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]

a halogen (e.g., F, Cl, Br) substituted compound thereof; or a pharmaceutically acceptable salt, tautomer, or solvate thereof.

In other embodiments, the RRmod can be an oxadiazole selected from:

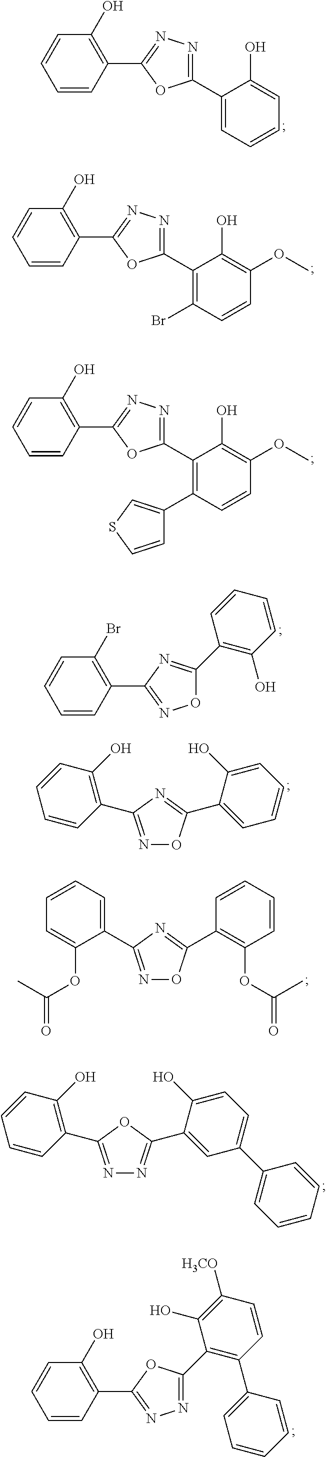

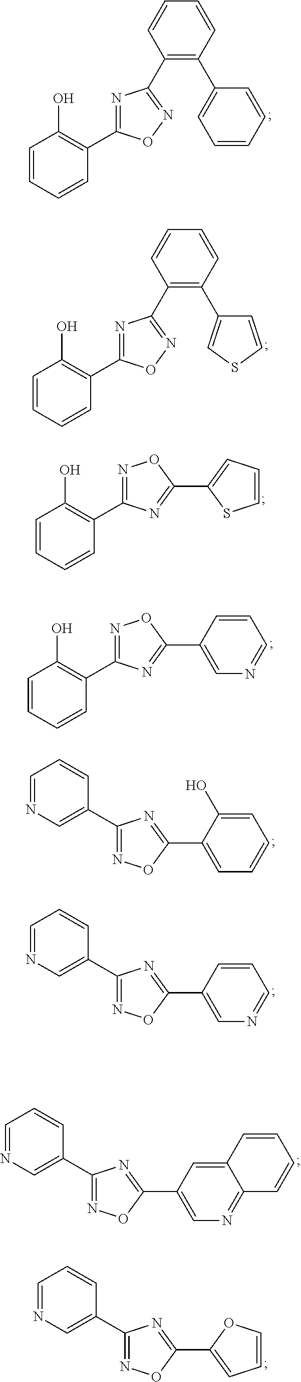

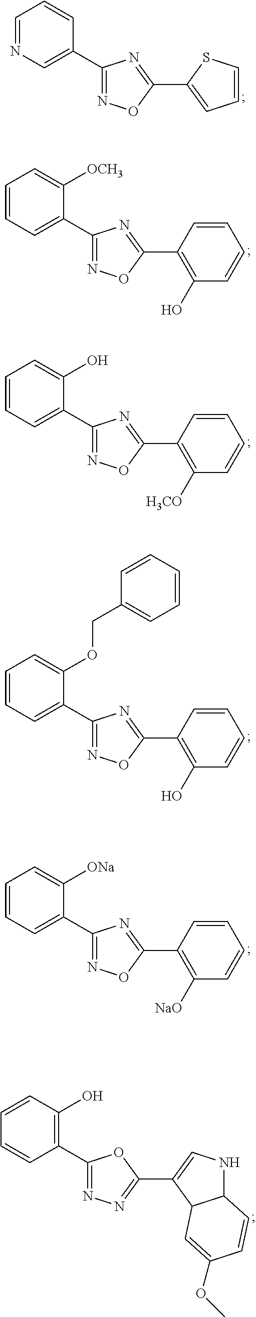

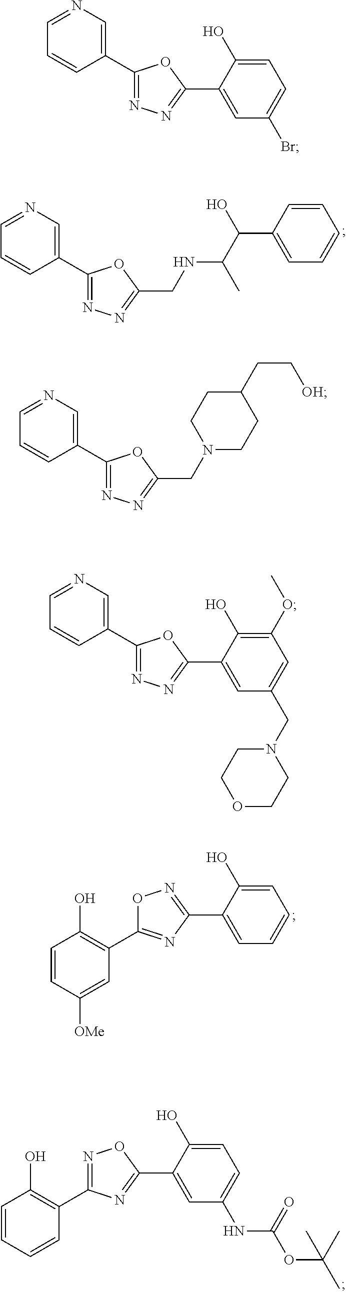 [SMILES: Oc1ccc(Br)cc1-c1nnc(-c2cccnc2)o1 ; CC(NCc1nnc(-c2cccnc2)o1)C(O)c1ccccc1 ; OCCC1CCN(Cc2nnc(-c3cccnc3)o2)CC1 ; COc1cc(CN2CCOCC2)cc(-c2nnc(-c3cccnc3)o2)c1O ; COc1ccc(O)c(-c2nc(-c3ccccc3O)no2)c1 ; CC(C)(C)OC(=O)Nc1ccc(O)c(-c2nc(-c3ccccc3O)no2)c1]

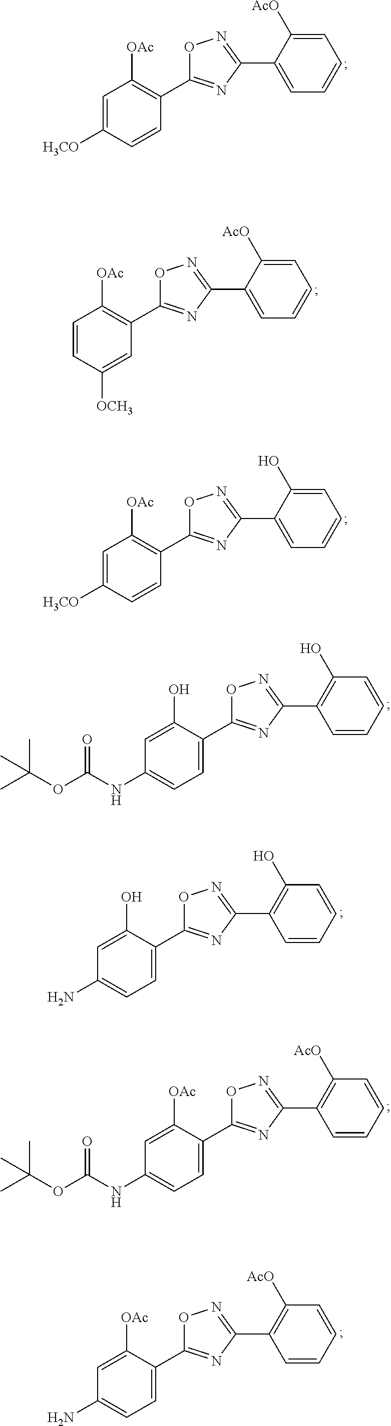

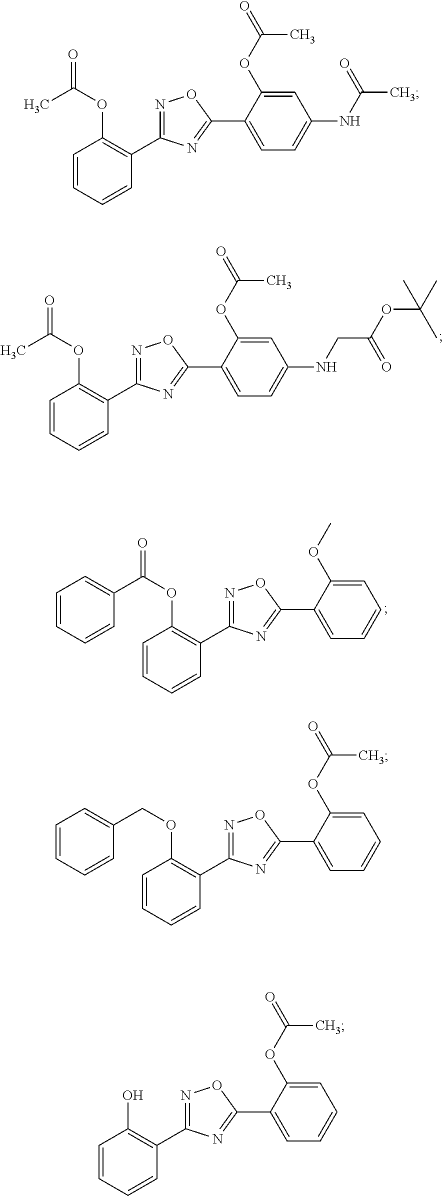 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]

a halogen (e.g., F, Cl, Br) substituted compound thereof; or a pharmaceutically acceptable salt, tautomer, or solvate thereof.

In other embodiments, the RRmod is a thiazole selected from:

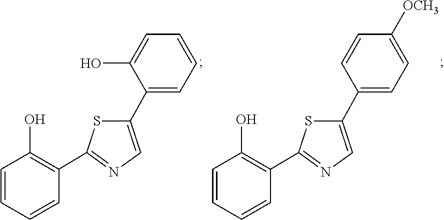 [SMILES: Oc1ccccc1-c1cnc(-c2ccccc2O)s1 ; COc1ccc(-c2cnc(-c3ccccc3O)s2)cc1]

a halogen (e.g., F, Cl, Br) substituted compound thereof; or a pharmaceutically acceptable salt, tautomer, or solvate thereof.

In other embodiments, the RRmod is a diazole, triazole, or tetrazole selected from:

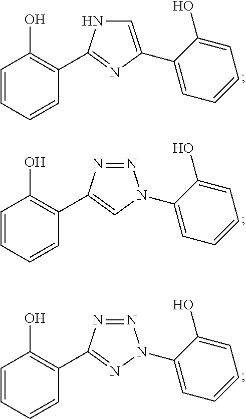

a halogen (e.g., F, Cl, Br) substituted compound thereof; or a pharmaceutically acceptable salt, tautomer, or solvate thereof.

Additional RRmods can be identified by screening compounds for the ability to modulate (e.g., inhibit or activate) ribonucleotide reductase enzyme activity. Candidate RRmods can be screened for function by a variety of techniques known in the art and/or disclosed within the instant application. Candidate compounds may be screened individually, in combination, or as a library of compounds.

Candidate compounds screened include chemical compounds. In some aspects, the candidate compound is a small organic molecule having a molecular weight of more than about 50 and less than about 2,500 Daltons. Compounds screened are also found among biomolecules including, but not limited to: peptides, saccharides, fatty acids, steroids, pheromones, purines, pyrimidines, derivatives, structural analogs or combinations thereof. The compounds screened can include functional groups necessary for structural interaction with proteins, particularly hydrogen bonding, and typically include at least an amine, carbonyl, hydroxyl, or carboxyl group.

Candidate compounds can be obtained from a wide variety of sources including libraries of synthetic or natural compounds. Compounds to be screened can be produced, for example, by bacteria, yeast or other organisms (e.g., natural products), produced chemically (e.g., small molecules, including peptidomimetics), or produced recombinantly. It is further contemplated that natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

In many drug screening programs, with test libraries of compounds and natural extracts, high throughput assays are desirable in order to maximize the number of compounds surveyed in a given period of time. Assays described herein may be developed with purified or semi-purified proteins or with lysates. These assays are often preferred as “primary” screens in that they can be generated to permit rapid development and relatively easy detection of an alteration in a molecular target, which is mediated by a test agent. Assays described herein can include cell-based assays. Cell-based assays may be performed as either a primary screen, or as a secondary screen to confirm the activity of compounds identified in a cell free screen, such as an in silico screen.

Embodiments described herein also relate to a method of screening in silico for a compound effective as an RRmod. For example, a 3-D model of the hexamer interface epitope of RR1 targeted by small molecules can be used to provide a pharmacophore using X-ray Crystallography. An initial model can then be generated using a suitable protein modeling software program. In some aspects, the model can then be subjected to energy refinement with a software program such as SURFLEX dock. The pharmacophore can be modified to comply to the Lipinski limits to design drug-like molecules with good bioavailability. In one embodiment, the template used for docking was the hexamer interface of ribonucleotide reductase as shown in FIG. 1.

Once a model is built, small molecule RRmods that bind to ribonucleotide reductase at the hexamer interface of RR1 can be identified by methods well known in the relevant art using in silico conformation screening techniques. For example, virtual screening of the University of Cincinnati Drug Discovery Center (UC DCC) Library of 350,000 compounds can be performed using the drug discovery software SYBYLX1.3 (Tripos, St. Louis, Mo.). Such software can also be used to design modified analogs of compounds for use as RRmods. In parallel, ZINC and other commercial databases can be searched using within SYBYLX1.3 software for lead compounds that satisfy the pharmacophore. These hits can be docked and scored using SURFLEX dock option in SYBYLX1.3. The best hits can then be discriminated using two scoring functions called, a docking score and the C-score. The docking score is theoretically equivalent to the negative logarithm of K_{d}, while C-score is a consensus scoring function. Hence, docking scores that are equal to 6 would mean a theoretical K_{d} of micromolar. The maximum C-score that can be obtained is five. Based on these criteria, after virtually screening the library, the best scoring candidates can be selected and then tested using various in vitro and cell based assays described herein and known in the art for efficacy. The larger numbers obtained for dock score and C-scores greater than 6 and 4-5 respectively represents the high ranking inhibitors that are predicted to have high affinities.

In some aspects, about 20,000 compounds can be selected from in silico screening for an in vitro high-throughput screening (HTS). HTS can be carried out using an automated HTS system which performs biochemical and cell-based assays using 96 or 384-well microtiter plates. The system includes detectors, CO₂ incubators, pipetting systems, a plate washer, centrifuge, a storage unit, bar code readers, xyz robots, turntables, and pushers necessary for fully automated screening. A Jobin Yvon-Spex fluorescence spectrophotometer can be used to record the spectra. Alternatively, a multimode PERKIN-ELMER plate reader can be used for detecting fluorescence intensity, fluorescence polarization, fluorescence resonance energy transfer, luminescence, or absorbance using ZEISS optics and a sensitive CCD camera. The PERKIN-ELMER Opera detector performs high content screening using confocal microscopy and image analysis software powered by onboard servers. Lasers and CCD cameras allow measurement of subcellular localization, binding events or any other microscopic images which can be rapidly quantitated. Image analysis is performed immediately after the image is captured and stored in a database. All other data can be analyzed using GENEDATA HTS analysis software (Switzerland), stored in a GENEDATA database based on ORACLE.

In some embodiments, in vitro HTS includes a fluorescence based assay adapted for HTS. For example, in vitro HTS can employ tryptophan fluorescence quenching. The binding sites of proteins are known to often contain tryptophan (Trp) residues, whose fluorescent properties may be altered upon ligand binding. Conformational changes within the binding site or simply the presence of the ligand can result in either fluorescence quenching or enhancement, which may be utilized to quantitatively investigate protein-ligand interactions. Change in intrinsic tryptophan fluorescence is used to measure the binding of a candidate agent to a targeted binding site of ribonucleotide reductase. The trytophan fluorescence spectra of Hurl (Human ribonucleotide reductase) and a candidate compound can be recorded and then compared in order to determine the extent of quenching. The ribonucleotide reductase samples can be titrated with 65 μM candidate compounds at room temperature where a decrease in fluorescence, or quenching, can be correlated with the binding affinity of the candidate compound to the targeted binding site of ribonucleotide reductase and/or a conformational change in the targeted ribonucleotide reductase binding site.

In some aspects, candidate RRmod compounds, including those collected from an in silico similarity search or HTS assay, may be further screened for efficacy using in vitro and/or in vivo experimental screening methods known in the art. The efficacy of an identified compound can be assessed by generating dose response curves from data obtained using various concentrations of the test compound. Moreover, a control assay can also be performed to provide a baseline for comparison. Such candidates can be further tested for their effects on cancer and tumor cell growth, proliferation, apoptosis, differentiation, and transformation properties compared to controls as well as their ability to: inhibit de novo DNA synthesis in vitro; unbalance nucleotide pool of DNA precursor molecules in vitro; modulate ribonucleotide reductase activity in vitro; and/or for other properties, such as the ability to inhibit cell growth and increase the toxicity of neoplastic cells in vivo.

In some embodiments, assays used for in vitro screening of candidate compounds for cell growth inhibition can include DNA synthesis assays and MTT colorimetric assays to measure cell metabolism. For example, a DNA synthesis assay can include the steps of: (a) contacting the neoplastic cell with various concentrations of a candidate compound; and (b) comparing the DNA synthesis of the cell in step (a) with the DNA synthesis of the cell in the absence of the compound so as to determine whether the compound significantly inhibits ribonucleotide reductase activity, thereby reducing the growth of the cell. One can also determine the IC₅₀ of a candidate compound if the compound is found to significantly inhibit ribonucleotide reductase activity. The IC₅₀ of a drug can be determined by constructing a dose-response curve and examining the effect of different concentrations of a candidate agent on cell growth and/or ribonucleotide reductase enzyme activity. IC₅₀ values can be calculated for a given compound by determining the concentration needed to inhibit half of the maximum biological response of the compound.

For in vivo screening of candidate compounds, the candidate compound can be administered in any manner desired and/or appropriate for delivery of the compound in order to affect a desired result. For example, the candidate compound can be administered to a mammalian subject by injection (e.g., by injection intravenously, intramuscularly, subcutaneously, or directly into the tissue in which the desired affect is to be achieved), topically, orally, or by any other desirable means.

Normally, this screen will involve a number of animals receiving varying amounts and concentrations of the candidate compounds (from no compound to an amount of compound that approaches an upper limit of the amount that can be delivered successfully to the animal), and may include delivery of the compound in different formulations. The compounds can be administered singly or can be combined in combinations of two or more, especially where administration of a combination of compounds may result in a synergistic effect.

The effect of compound administration upon the animal model can be monitored by any suitable method such as assessing the number and size of tumors, overall health, survival rate, etc. A candidate compound is identified as an effective compound for use in the treatment of a neoplastic disorder in a subject where candidate compound inhibits neoplastic cell growth in the animal in a desirable manner (e.g., by binding to the Sml1 allosteric binding site of ribonucleotide reductase and allosterically inhibiting the enzyme's activity, etc.). In some aspects, effective compounds can be identified as having low toxicity in vivo.

RRmods disclosed herein have been shown to bind to epitopes (e.g., M-site or C-site) of the large α-subunit of RR1 and inhibit growth of multiple cancer cell types in vitro, supporting the use of these RRmods to treat a wide range of neoplastic diseases and disorders. Thus, in accordance with another embodiment, RRmods described herein can be used for the preparation of a pharmaceutical composition for the treatment of a neoplastic disorder in a subject. In one embodiment, the subject is suffering from a neoplastic disorder characterized by increased cell growth. In another embodiment, the subject is suffering from cancer.

A therapeutically effective amount of an RRmod described herein can be administered to a subject for the treatment of a variety of conditions in order to inhibit cell growth in the subject. Such conditions include, without being limited thereto, neoplastic disorder, and in particular all types of solid tumors; skin proliferative diseases (e.g., psoriasis); and a variety of benign hyperplasic disorders.

In one aspect, the neoplastic disorder is cancer. The cancer can include, but is not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, meningoma, medulloblastoma, schwannoma and epidymoma. In certain aspects, the cancer is a pancreatic, breast, lung, colon or glioblastoma cancer.

In another aspect, the neoplastic disorder is a solid tumor. Exemplary solid tumors include carcinomas, sarcomas, adenomas, and cancers of neuronal origin and if fact to any type of cancer which does not originate from the hematopoietic cells and in particular concerns: carcinoma, sarcoma, adenoma, hepatocellular carcinoma, hepatocellularcarcinoma, hepatoblastoma, rhabdomyosarcoma, esophageal carcinoma, thyroid carcinoma, ganglioblastoma, fibrosarcoma, myxosarcoma, liposarcoma, cohndrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all the above.

Benign hyperplasic disorders include, without being limited thereto, benign prostate hyperplasia (BPH), non-tumorigenic polyps in the digestive tract, in the uterus and others.

In addition to cancer, the RRmods disclosed herein may be used to treat other conditions associated with aberrant ribonucleotide reductase enzyme activity such as for example various mitochondrial, redox-related, degenerative diseases, and viruses such as HIV.

When used as therapeutic agents in the treatment of neoplastic disorders, the RRmods can be conveniently formulated into pharmaceutical formulations composed of one or more of the compounds (e.g., RRmods of formulas (I-V) or an RRmod identified by a screening assay as described above) in association with a pharmaceutically acceptable carrier or excipient. (See Remington: The Science and Practice of Pharmacy (Gennaro ed. 22nd Edition, Pharmaceutical Press, London, UK, 2012), which discloses typical carriers and conventional methods of preparing pharmaceutical formulations).

In making the compositions, the RRmod is usually mixed with the excipient, diluted by an excipient or enclosed within a carrier which can be in the form of a capsule, sachet, paper or other container. When the excipient serves as a diluent, it can be a solid, semi-solid, or liquid material, which acts as a vehicle, carrier or medium for the RRmod. Thus, the compositions can be in the form of tablets, pills, powders, lozenges, sachets, cachets, elixirs, suspensions, emulsions, solutions, syrups, aerosols (as a solid or in a liquid medium), soft and hard gelatin capsules, suppositories, sterile injectable solutions, and sterile packaged powders. The RRmods can also be administered to a subject as a stabilized prodrug to increase the activity, bioavailability, stability or otherwise alter the properties of the RRmod.

The effective amount of RRmod in the pharmaceutical composition and unit dosage form thereof may be varied or adjusted widely depending upon the particular application, the manner or introduction, the potency of the particular compound, and the desired concentration.

The effective amount is typically determined in appropriately designed clinical trials (dose range studies) and the person versed in the art will know how to properly conduct such trials in order to determine the effective amount. As generally known, an effective amount depends on a variety of factors including the affinity of the RRmod to the targeting binding site (e.g., the M-site or C-site of hRRM1), its distribution profile within the body, a variety of pharmacological parameters such as half life in the body, on undesired side effects, if any, on factors such as age and gender, etc.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In this case, the composition will typically be administered over an extended period of time in a single daily dose, in several doses a day, as a single dose and in several days, etc. The treatment period will generally have a length proportional to the length of the disease process and the specific RRmod effectiveness and the patient species being treated.

RRmods and pharmaceutical compositions thereof can be administered to the subject by any suitable means, including, for example, oral, intravenous, intramuscular, intra-arterial, subcutaneous, intranasal, via the lungs (inhalation) and through local administration.

RRmods described herein can be used as single agents or in combination or in conjunction with one or more other therapeutic agents in the treatment of the aforementioned diseases, disorders and conditions for which RRmods or the other agents have utility. In some embodiments, a combination of an RRmod and other therapeutic agent together is safer or more effective than either drug alone.

In some embodiments, the other therapeutic agent used in a combination therapy can include at least one anti-proliferative agent selected from the group consisting at least one of a chemotherapeutic agent, an anticancer agent, an antimetabolite, a DNA damaging agent, an antitumorgenic agent, an antimitotic agent, an antiviral agent, an antineoplastic agent, an immunotherapeutic agent, and a radiotherapeutic agent. Additional therapeutic agents used in combination therapies with RRmods can include biguanides (e.g., metformin, phenformin and buformin), AP endonuclease inhibitors (e.g., methoxyamine (MX)), BER inhibitors including PARP inhibitors, and ribonucleotide reductase inhibiting agents. Exemplary ribonucleotide reductase inhibiting agents for use in conjunction with RRmods include 2′,2′-difluoro 2′ deoxycytidine (gemcitabine), O⁶-methyl-arabinofuranosyl guanine (nelarabine), 2′-fluro-2′-deoxyarabinofuranosyl-2-chloroadenine (clofarabine), N⁴-pentyloxycarbonyl-5′-deoxy-5-flurocytidine (capecitabine), 2,2-difluoro-2′-deoxyadenosine (cladribine), arabinofuranosyl-2-fluoroadenine (fludarabine), 2′-deoxycoformycin (pentostatin), 5-fluro-2′deoxyuridine, arabinofuranosylcytosine (cytarabine), 6-thioguanine, 5-fluorouracil, methotrexate, 6-mercaptopurine.

In some aspects, RRmods can be used in a combination therapy with an anti-proliferative agent. The phrase “anti-proliferative agent” can include agents that exert antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development, which can be included by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endotheliai cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

In the instances of combination therapies described herein, it will be understood the administration further includes a pharmaceutically or therapeutically effective amount of the additional therapeutic agent in question. The second or additional therapeutic agents described herein may be administered in the doses and regimens known in the art or may be administered in low doses.

In some embodiments, the administration of a RRmod and an additional therapeutic agent can result in a synergistic effect. A “synergistic effect” as used herein means the combined effect of two or more therapeutic agents can be greater than the sum of the separate effects of the agents alone. For example, the combined effect of an RRmod, and an anticancer agent, such as metformin or another RRmod such as gemcitabine, can be greater than the sum of the separate effects of a single RRmod and metformin or gemcitabine alone.

In some embodiments, the combined effect of administering two or more RRmod compounds is greater than the sum of the separate effects of the RRmods alone. In certain embodiments, a NSAAH hydrazone RRmod, as described in US Patent Application US2016/065928, the subject matter of which is incorporated herein by reference in its entirety, can be administered in combination with one or more oxadiazole, thiazole, diazole, triazole, or tetrazole RRmods describe herein to produce a synergistic therapeutic effect. In a particular embodiment, a NSAAH RRmod and an oxadiazole, thiazole, diazole, triazole, or tetrazole RRmod described herein can be administered in combination to produce a synergistic therapeutic effect.

Where the combined effect of administering a RRmod and another therapeutic agent is greater than the sum of the separate effects of the RRmod and the other agent alone, the RRmod and/or therapeutic agent can be administered to the subject in a lower dose or even a sub-therapeutic dose. A benefit of lowering the dose of the combination therapeutic agents and therapies can include a decrease in the incidence of adverse effects associated with higher dosages. For example, by the lowering the dosage of a chemotherapeutic agent such as methotrexate, a reduction in the frequency and the severity of nausea and vomiting will result when compared to that observed at higher dosages.

The additional therapeutic agent can be administered by a route and in an amount commonly used therefore, contemporaneously or sequentially with a RRmod compound. When administered as a combination, a RRmod compound and additional therapeutic agent(s) can be formulated as separate compositions which are given at the same time or different times, or the therapeutic agents can be given as a single composition.

In other embodiments, the RRmods described herein can be used to treat cancer that is resistant or has acquired resistance to anticancer agents, such as antimitotic agent and other RRmods. For example, the RRmods can be used to treat cancer that is resistant or has acquired resistance to treatment with gemcitabine.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Reversible Thiazole-Based RR Modulators Inhibiting the Catalytic Site for the Treatment of Cancer

Ribonucleotide reductase is a multi-protein enzyme consisting of a large subunit called hRRM1 containing the catalytic site and allosteric sites and a small subunit called hRRM2 that houses the free radical required for initiating radical-based chemistry (FIG. 1A).

The hRRM1 subunit catalyzes the conversion of four ribonucleoside diphosphates (UDP, CDP, GDP and ADP) to their respective deoxy forms. During the S-phase of the cell cycle, these reduction reactions are allosterically controlled by binding of nucleotide triphosphates to two different sites on RR (Brown and Reichard and others). The S-site is located at the dimer interface of hRRM1 and is involved in allosterically regulating substrate binding specificity (FIG. 1A). ATP activates the enzyme by binding at the A-site while dATP inactivates the enzyme by binding at the A-site. (FIG. 1A).

Recent studies with RR have revealed the importance of oligomerization and its regulation. By convention the hRRM1 subunit is referred to as a and the hRRM2 subunit as β. Although the multimerization of RR is still a subject of investigation, the prevailing model is that RR minimally functions as an α₂β₂ complex. At physiological concentrations of ATP (3 mM) RR exists predominantly as a hexamer with a small population of dimer present. When dATP is bound, the large subunit has been shown to exist as a dimer and hexamer, while baculovirus expressed mouse RR1 was observed to exist as a tetramer. Recently, hexamer formation has been shown to be important for drugs such as gemcitabine and clofarabine binding to RR. For example, gemcitabine was shown to inactivate hRRM1 by inducing α6β6 oligomers while clofarabine was shown to bind hRRM1 hexamers with nanomolar affinity. While this drug was shown to induce hRRM1 dimers, it is unable to induce the formation of hexamers, leading the authors to conclude that 5-NINTP loses its inhibitory potency due to its inability to form hexamers.

We previously characterized the hydrazone compound NSAAH (Naphthyl Salicyl Acyl Hydrazone) that inhibits hRR reversibly with micromolar affinity in vitro. The crystal structure of the NSAAH complex with hRR together with steady state kinetic data demonstrated that it binds in the C-site of hRRM1 (See FIG. 1). Importantly, the IC₅₀ for NSAAH was within two fold of that of Gemcitabine for growth inhibition of multiple cancer cell lines. However, NSAAH demonstrated little measurable cytotoxicity against normal mobilized peripheral blood progenitor cells. Thus, these data identified the first non-nucleoside competitive inhibitor of hRRM1, and reveal its improved in vivo inhibition properties relative to existing therapeutics providing a starting point for rational fragment-based drug design of a new class of hRR inhibitors.

We examined the structure of the hydrazone bound to the catalytic site (C-site) of RR1. The mode of binding of the hydrazone moiety was postulated to be replaced by a thiazole as the geometry and stereochemistry would be retained. Furthermore, the thiazole permits multiple substitutions to create diversity libraries. The hypothesis was tested by designing and modeling several thiazole derivatives using the docking software Schrödinger, which predicted favorable binding to the hRRM1 active site. Initially, two thiazole derivatives were synthesized and tested using the RR radioactivity assay for inhibition. Table 1 show the initial data.

In a second series of experiments the hydrazone was replaced by an oxadiazole, thiazole, diazole, triazole, or tetrazole five-membered ring that is also an excellent replacement. A small library consisting of the phenol or bromobenzene moiety conjugated to the oxadiazole, thiazole, diazole, triazole, or tetrazole followed by benzene substitutions were synthesized and tested in cell growth inhibition assays against the HTC116 cell line and the Panc1 cell line (see Table 2). It is anticipated that a few of the compounds tested have activity in the low micromolar range.

TABLE 1
Identification of two novel hRRM1 inhibitors using in silico docking,
and growth inhibition

| Structure | Activity |
|---|---|
| 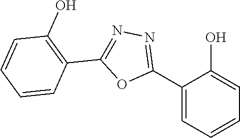 [SMILES: Oc1ccccc1-c1nnc(-c2ccccc2O)o1] | *** |
| 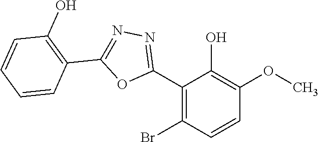 [SMILES: COc1ccc(Br)c(-c2nnc(-c3ccccc3O)o2)c1O] | *** |
| 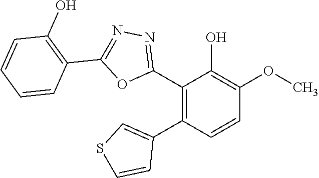 [SMILES: COc1ccc(-c2ccsc2)c(-c2nnc(-c3ccccc3O)o2)c1O] | *** |
| 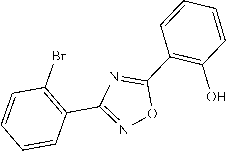 [SMILES: Oc1ccccc1-c1nc(-c2ccccc2Br)no1] | **** |
| 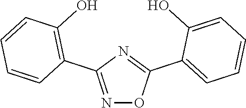 [SMILES: Oc1ccccc1-c1noc(-c2ccccc2O)n1] | ***** |
| 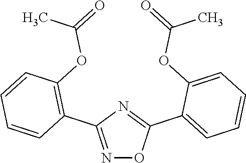 [SMILES: CC(=O)Oc1ccccc1-c1noc(-c2ccccc2OC(C)=O)n1] | ***** |
| 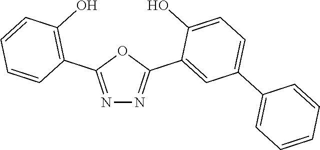 [SMILES: Oc1ccccc1-c1nnc(-c2cc(-c3ccccc3)ccc2O)o1] | *** |
| 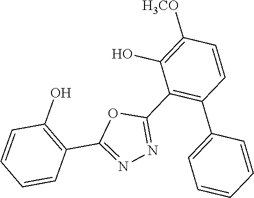 [SMILES: COc1ccc(-c2ccccc2)c(-c2nnc(-c3ccccc3O)o2)c1O] | *** |
| 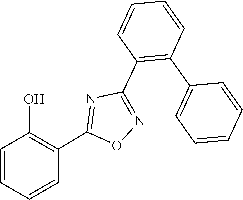 [SMILES: Oc1ccccc1-c1nc(-c2ccccc2-c2ccccc2)no1] | *** |
| 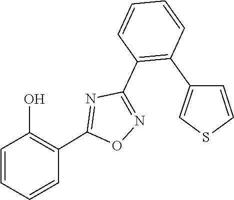 [SMILES: Oc1ccccc1-c1nc(-c2ccccc2-c2ccsc2)no1] | *** |
| 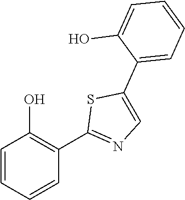 [SMILES: Oc1ccccc1-c1cnc(-c2ccccc2O)s1] | *** |
| 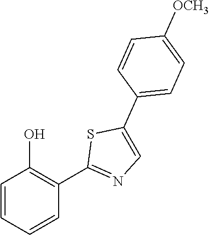 [SMILES: COc1ccc(-c2cnc(-c3ccccc3O)s2)cc1] | *** |
| 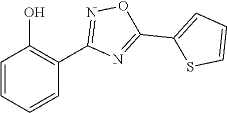 [SMILES: Oc1ccccc1-c1noc(-c2cccs2)n1] | *** |
| 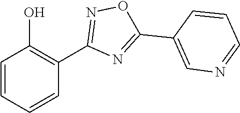 [SMILES: Oc1ccccc1-c1noc(-c2cccnc2)n1] | **** |
| 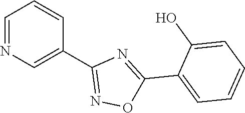 [SMILES: Oc1ccccc1-c1nc(-c2cccnc2)no1] | **** |
| 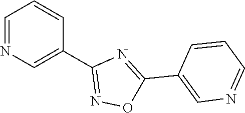 [SMILES: c1cncc(-c2noc(-c3cccnc3)n2)c1] | **** |
| 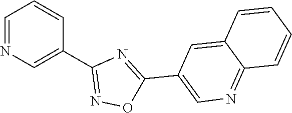 [SMILES: c1cncc(-c2noc(-c3cnc4ccccc4c3)n2)c1] | *** |
| 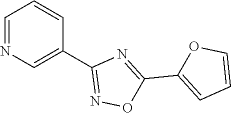 [SMILES: c1cncc(-c2noc(-c3ccco3)n2)c1] | *** |
| 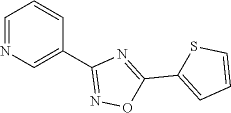 [SMILES: c1cncc(-c2noc(-c3cccs3)n2)c1] | *** |
| 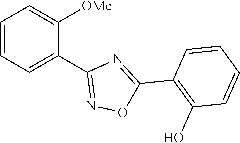 [SMILES: COc1ccccc1-c1noc(-c2ccccc2O)n1] | *** |
| 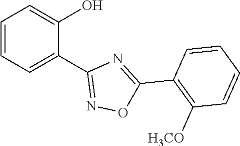 [SMILES: COc1ccccc1-c1nc(-c2ccccc2O)no1] | **** |
| 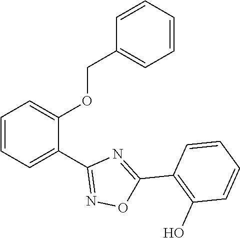 [SMILES: Oc1ccccc1-c1nc(-c2ccccc2OCc2ccccc2)no1] | *** |
| 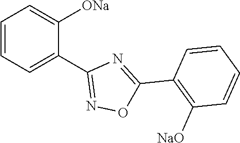 | *** |
| 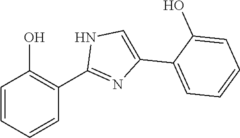 [SMILES: Oc1ccccc1-c1c[nH]c(-c2ccccc2O)n1] | *** |
| 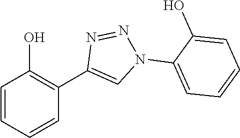 [SMILES: Oc1ccccc1-c1cn(-c2ccccc2O)nn1] | *** |
| 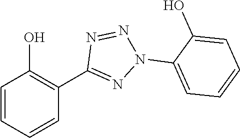 [SMILES: Oc1ccccc1-c1nnn(-c2ccccc2O)n1] | *** |
| 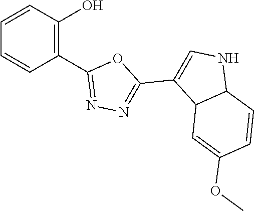 [SMILES: COC1=CC2C(c3nnc(-c4ccccc4O)o3)=CNC2C=C1] | ** |
| 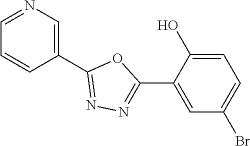 [SMILES: Oc1ccc(Br)cc1-c1nnc(-c2cccnc2)o1] | *** |
| 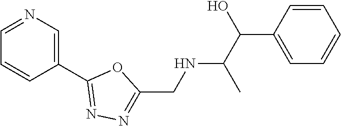 [SMILES: CC(NCc1nnc(-c2cccnc2)o1)C(O)c1ccccc1] | ** |
| 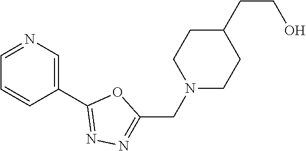 [SMILES: OCCC1CCN(Cc2nnc(-c3cccnc3)o2)CC1] | ** |
| 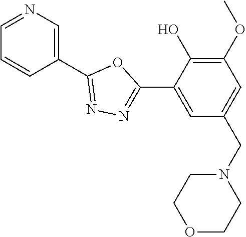 [SMILES: COc1cc(CN2CCOCC2)cc(-c2nnc(-c3cccnc3)o2)c1O] | ** |
| 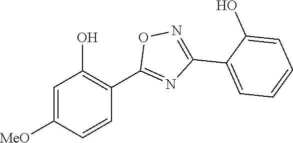 [SMILES: COc1ccc(-c2nc(-c3ccccc3O)no2)c(O)c1] | |
| 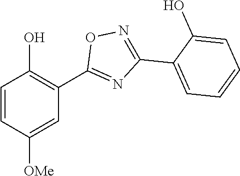 [SMILES: COc1ccc(O)c(-c2nc(-c3ccccc3O)no2)c1] | |
| 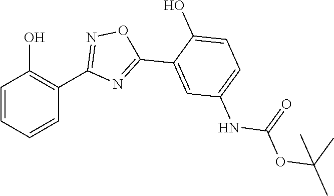 [SMILES: CC(C)(C)OC(=O)Nc1ccc(O)c(-c2nc(-c3ccccc3O)no2)c1] | |
| 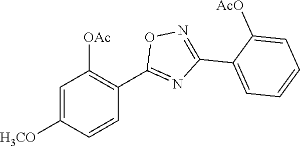 [SMILES: COc1ccc(-c2nc(-c3ccccc3OC(C)=O)no2)c(OC(C)=O)c1] | |
| 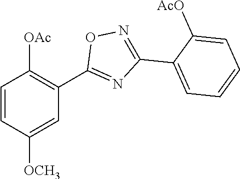 [SMILES: COc1ccc(OC(C)=O)c(-c2nc(-c3ccccc3OC(C)=O)no2)c1] | |
| 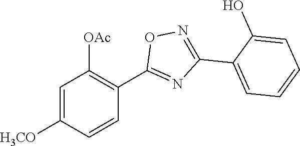 [SMILES: COc1ccc(-c2nc(-c3ccccc3O)no2)c(OC(C)=O)c1] | |
| 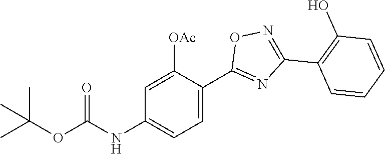 [SMILES: CC(=O)Oc1cc(NC(=O)OC(C)(C)C)ccc1-c1nc(-c2ccccc2O)no1] | |
| 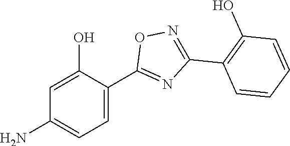 [SMILES: Nc1ccc(-c2nc(-c3ccccc3O)no2)c(O)c1] | |
| 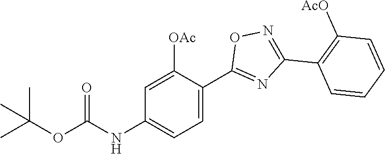 [SMILES: CC(=O)Oc1ccccc1-c1noc(-c2ccc(NC(=O)OC(C)(C)C)cc2OC(C)=O)n1] | |
| 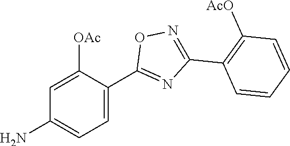 [SMILES: CC(=O)Oc1ccccc1-c1noc(-c2ccc(N)cc2OC(C)=O)n1] | |
| 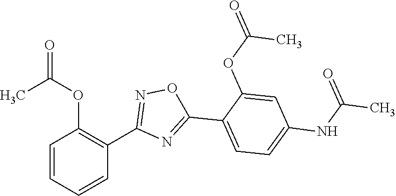 [SMILES: CC(=O)Nc1ccc(-c2nc(-c3ccccc3OC(C)=O)no2)c(OC(C)=O)c1] | |
| 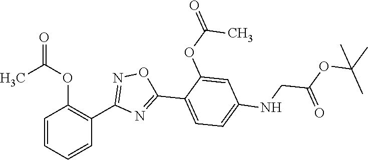 [SMILES: CC(=O)Oc1ccccc1-c1noc(-c2ccc(NCC(=O)OC(C)(C)C)cc2OC(C)=O)n1] | |
| 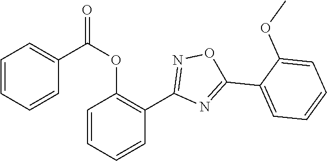 [SMILES: COc1ccccc1-c1nc(-c2ccccc2OC(=O)c2ccccc2)no1] | |
| 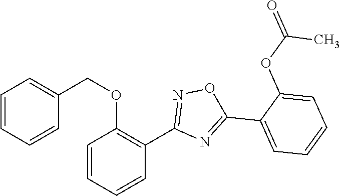 [SMILES: CC(=O)Oc1ccccc1-c1nc(-c2ccccc2OCc2ccccc2)no1] | |
| 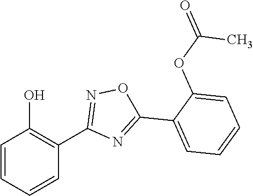 [SMILES: CC(=O)Oc1ccccc1-c1nc(-c2ccccc2O)no1] | |

Activity measurements in Table 2 is performed as described below. Briefly, six concentrations of the compounds ranging from 1-100 μM of the compounds are incubated on ice with 2.5 μmol of hRRM1 for 30 minutes. The assay sample was then diluted by a factor of 5, and enzyme activity was assayed in triplicate. As a control, the assay is also performed for non-drug-treated hRRM1 and for hRRM1 with 50 μM NSAAH without dilution. Activity measurements are based on the qualitative relation between the minimum concentration of compound required to produce inhibit enzyme activity. Potency activities are ranked as either extremely high, very high or significant. These activities are used to determine the activity ranking. Compounds which are found to have extremely high potency are classified as “****” Compounds which are found to have very high potency of protein synthesis are classified as “***”. Compounds which are found to have significant potency are classified as

### Material and Methods

### Thiazole Synthesis

Thiazoles are synthesized by replacing hydrazone moieties with a thiazole as previously described using well known methods.

### Oxadiazole Synthesis

Oxadiazoles are produced by refluxing acyl hydrazones in DMSO in the presence of excess iodine (1.2 equivalents) and K₂CO₃ (3 equivalents). The reaction is diluted by 20 in 5% N₂S₂O₃(aq), then extracted with ethyl acetate (5×20 mLs). The organic solutions are back extracted with water (3×15 mLs), dried by MgSO₄, filtered, and concentrated en vacuo to collect the crude product. Compounds are purified by flash column chromatography (silica gel, 10% Ethyl Acetate/Hexanes).

Protein Expression and Purification of hRRM1

The hRRM1 protein is expressed in E. coli BL21 DE3 (RIL) and purified using peptide affinity chromatography. The homogeneous protein is pooled and concentrated to 20-25 mg/ml, as quantified by UV absorbance spectroscopy.

### Establishing Reversible Inhibition of hRR

In assay buffer, six concentrations of the compounds ranging from 1-100 μM of the compounds are incubated on ice with 2.5 μmol of hRRM1 for 30 minutes. The assay sample was then diluted by a factor of 5, and enzyme activity was assayed in triplicate. As a control, the assay was also performed for non-drug-treated hRRM1 and for hRRM1 with 50 μM NSAAH without dilution.

### Crystallization and Data Collection

20 mg/ml hRRM1 protein is incubated with 20 mM dTTP and 1 mM NSAAH at 4° C. for 30 minutes. The drop consisting of hRRM1-TTP-NSAAH is cross-seeded with preformed hRRM1-dTTP crystals previously reported in reference to form the co-crystal of hRRM1-TTP-NSAAH. The crystals are screened by the hanging drop method. The well solution for crystallization is composed of 100 mM Tris, pH 7.9, 200 mM Li₂SO₄, and 19% PEG-3350. Diffraction quality crystals appears after one week and which is transferred to the mother liquor, with 20% glycerol as cryo-protectant, and then flash frozen in liquid nitrogen for data collection. Diffraction data are collected with crystals flash cooled at 100 K in a stream of liquid N₂ using a synchrotron radiation source, NECAT beamline, at APS (Advanced Photon Source) Chicago. The crystals are of space group P2₁2₁2₁ and diffracted to 2.66 Å resolution.

### Structure Solution and Refinement

The structure of the NSAAH-hRRM1 complex structure was solved by molecular replacement using a previously solved structure of hRRM1-GDP-dTTP (PDB ID 3HNC) as a search model. Molecular replacement gave a single prominent solution after rotation and translation function. The initial solution was refined by rigid body refinement, which produced a clearly interpretable electron density map for the overall structure. The ligand density for NSAAH was clearly observed at the active site of hRRM1. Manual adjustment of the backbone and sidechain of the model was conducted in Coot. Crystallographic refinement was carried out using the programs Phenix and refmac 5 within CCP4 suite. Difference Fourier maps with coefficients 2|Fₒ|-|F_{c}| and |Fₒ|-|F_{c}| were used to model NSAAH interacting with amino acid residues at the catalytic site. After a few rounds of model building water molecules were added using the |Fₒ|-|F_{c}| map peaks above 3.0σ. The value of R_{free} can be used as an indicator to validate the water picking and refinement and to avoid any possible over fitting of the data.

### Cell-Free Inhibition Studies

The IC₅₀ are determined using, boronate chromatography. Six concentrations of the RRmod are studied ranging from 1-100 μM. The assay is repeated in triplicate. Data are fitted in GraphPad Prism 6.05 using a sigmoidal dose-response curve.

### Binding Studies Using Fluorescence Quenching

To assay binding the compounds are titrated into a solution of 0.5 mg hRRM1 at 1-200 μM in 10 μM increments. The emission spectrum is recorded over 300-400 nm using a Jobin Yvon-Spex fluorescence spectrophotometer. The data is fitted by nonlinear regression using the one-site binding (hyperbola) equation Y=Bmax*X/(Kd(app)+X), where Bmax is the maximum extent of quenching and Kd(app) is the apparent dissociation constant, using GraphPad Prism 6.05. Measurements were recorded in duplicate.

### Blood Progenitor Colony Forming Unit (CFU) Assay

The CFU assay can be performed in the Hematopoietic Biorepository and Cellular Therapy Core Facility of the Case Comprehensive Cancer Center using a standardized protocol.

Briefly, mobilized peripheral blood mononuclear cells are collected from healthy donors by apheresis after Neupogen stimulation under University Hospitals IRB Protocol #09-90-195. Excess discarded cells are diluted to 1(10⁶) cells/mL in RPMI1640+15% fetal bovine serum, 100 U/mL penicillin, 100 μg/mL streptomycin and 50.4 units/ml GM-CSF. Drug solution is added to the cell suspension and 9× volume of complete methylcellulose (Methocult H4434+50 μM hemin). The methylcellulose/cell suspension was aliquoted into triplicate 35-mm gridded tissue culture plates and incubated in a humidified 5% CO₂ incubator at 37° C. for 14 days. Plates were counted visually and clusters of >50 cells were scored as surviving colonies.

### Cancer Cell Line Growth Inhibition Assay

The growth inhibition assay can be performed in the Preclinical Drug Testing Laboratory of the Case Comprehensive Cancer Center using a standardized protocol. Cell lines (human colon cancer HCT116 and human pancreas ductal adenocarcinoma Panc1 cells) re maintained in standard growth media; RPMI1640+10% FBS+2 mM glutamine+100 U/ml penicillin, 100 μg/ml streptomycin and shown to be negative for mycoplasma contamination using the MycoAlert™ Mycoplasma Detection kit (Lonza, Basel, Switzerland). Cell identity was verified by Short Tandem Repeat (STR) testing performed using the Promega StemElite kit, in the Genetic Resources Core Facility (GRCF) at Johns Hopkins University.

For growth inhibition assays, cells are harvested by trypsinization and seeded into 96-well tissue culture plates at 2500 cells/mL. The following day an equal volume of 2×-drug containing medium is added to each well. The cells are cultured for 3 additional days at 37° C. in a 5% CO₂ humidified incubator. Cell growth is assessed by measuring DNA content per well using the method of Labarca and Paigen. Dye fluorescence is measured in a Perkin-Elmer 1420 Victor3 Multilabel plate reader using 355 nm excitation and 460 nm emission.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A method of modulating ribonucleotide reductase activity in a neoplastic cell comprising administering to the cell an amount of a ribonucleotide reductase allosteric modulator (RRmod), the RRmod comprising a thiazole, an oxadiazole, or analog thereof that is administered at an amount effective to inhibit neoplastic cell growth.

2. The method of claim 1, wherein the RRmod includes a include compound having the following formula (I): 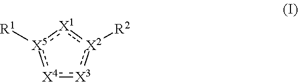
or a pharmaceutically acceptable salt, tautomer, or solvate thereof;
wherein:
X¹, X³, and X⁴ are each independently C, S, O, or N;
X² and X⁵ are each independently C or N;
at least two but no more than four of X¹, X², X³, X⁴, or X⁵ is not C;
---- is an optional bond;
R¹ and R² are each independently an alkyl, aryl, heteroaryl, or heterocyclyl optionally substituted with one or more R⁴; and
each R⁴ is independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety, and wherein R¹ and R² are not a naphtholyl.

3. The method of claim 2, wherein at least one of X¹, X³, or X⁴ is N.

4. The method of claim 2, wherein at least one of X² or X⁵ is C.

5. The method of claim 2, wherein X¹, X², X³, X⁴, and X⁵ define an oxadiazole, thiazole, diazole, triazole, or tetrazole.

6. The method of claim 2, R⁴ is a halogen, —OH, C₁-C₆ alkyl, alkoxy, alkoxycarbonyl, aryl, heterocyclyl, or heteroaryl.

7. The method of claim 2, wherein R¹ and R² are each independently selected from the group consisting of: 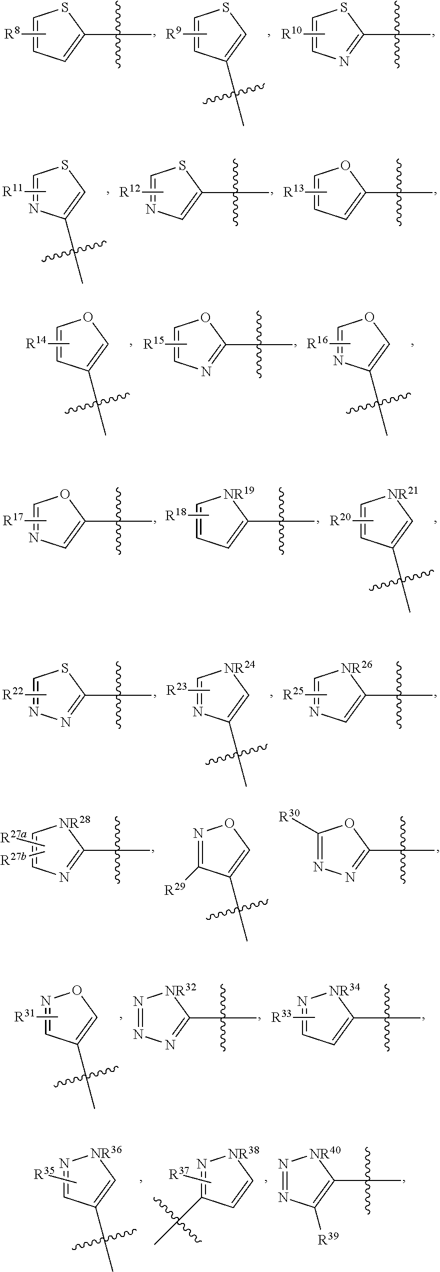 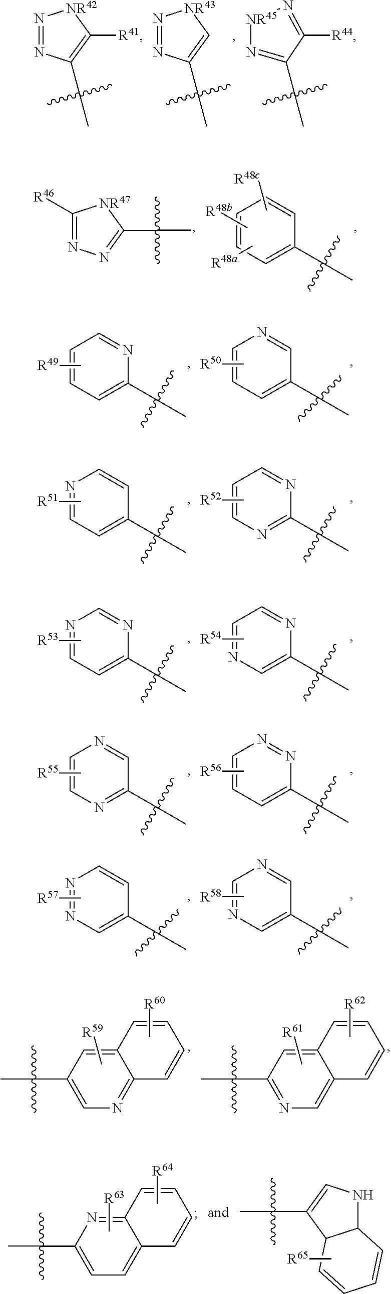
R⁸, R⁹, R¹⁰, R¹³, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, and R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

8. The method of claim 2, wherein R¹ and R² are each independently a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

9. The method of claim 2, wherein R² is selected from the group consisting of: 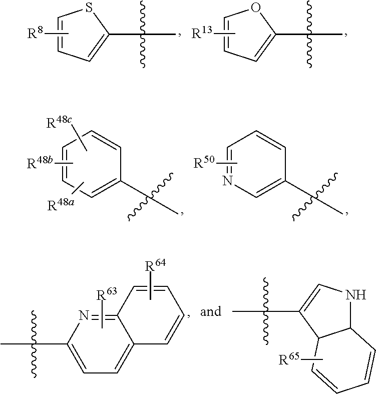
wherein R⁸, R¹³, R^{48a}, R^{48b}, R^{48c}, R⁵⁰, R⁶³, R⁶⁴ and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

10. A method of modulating ribonucleotide reductase activity in a neoplastic cell, the method comprising administering to the cell an amount of a compound having the following formula (III): 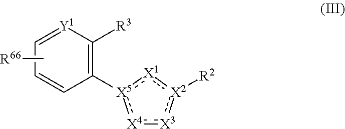
or a pharmaceutically acceptable salt, tautomer, or solvate thereof, wherein:
wherein:
X¹, X³, and X⁴ are each independently C, S, O, or N;
X² and X⁵ are each independently C or N;
at least two but no more than four of X¹, X², X³, X⁴, or X⁵ is not C;
Y¹ is Nor C;
---- is an optional bond;
R² is an alkyl, aryl, heteroaryl, or heterocyclyl optionally substituted with one or more R⁴;
R³ and R⁴ are each independently selected from a halogen or optionally substituted alkyl, alkenyl, alkynyl, hydroxyl, acetate, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;
R⁶⁶ is absent, halogen, alkoxy, or amino; and
R² is not a naphtholyl.

11. The method of claim 10, wherein at least one of X¹, X³, or X⁴ is N.

12. The method of claim 10, wherein at least one of X² or X⁵ is C.

13. The method of claim 10, wherein X¹, X², X³, X⁴, and X⁵ define an oxadiazole, thiazole, diazole, triazole, or tetrazole.

14. The method of claim 18, wherein R³ is halogen, —OH, alkyl, alkoxy, —O— alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

15. The method of claim 10, wherein R² is independently selected from the group consisting of: 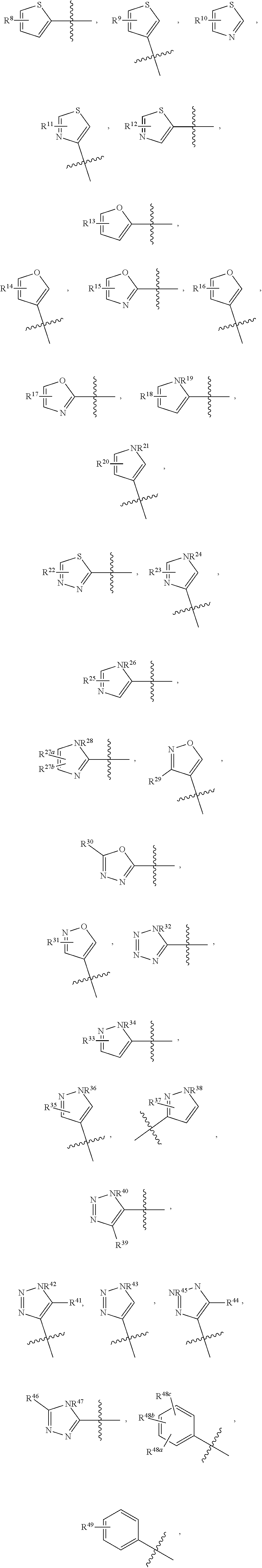 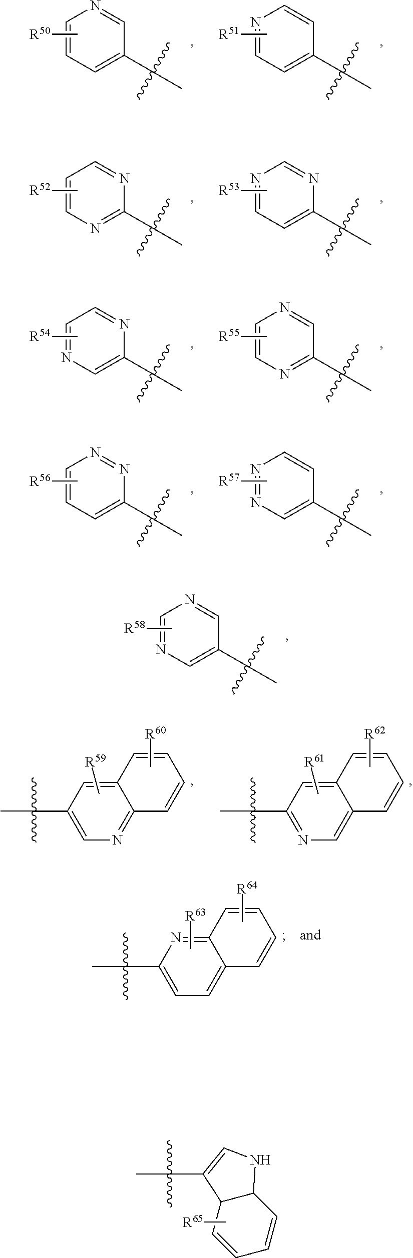
R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R^{48a}, R^{48b}, R^{48c}, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵ are each independently absent, halogen, or an optionally substituted hydroxyl, acetate, alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

16. The method of claim 10, wherein R² is a thiophenyl, furanyl, phenyl, pyridinyl, pyridazinyl, pyrimidinyl, triazinyl, quinolinyl, or indolyl each of which is optionally substituted with one or more halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

17. The method of claim 10, wherein R² is selected from the group consisting of: 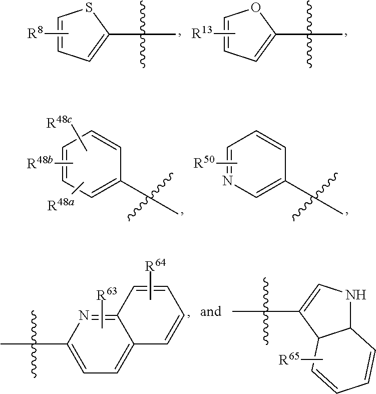
wherein R⁸, R¹³, R^{48a}, R^{48b}, R^{48c}, R⁵⁰, R⁶³, R⁶⁴, and R⁶⁵ are each independently absent, halogen, —OH, alkyl, alkoxy, —O-alkylene-aryl, alkoxycarbonyl, —O—(CO)-alkyl, —O—(CO)-aryl, amino, —N—(CO)-alkyl, —N—(CO)O-alkyl, aryl, heterocyclyl, or heteroaryl.

18. The method of claim 1, wherein the RRmod is a compound selected from: 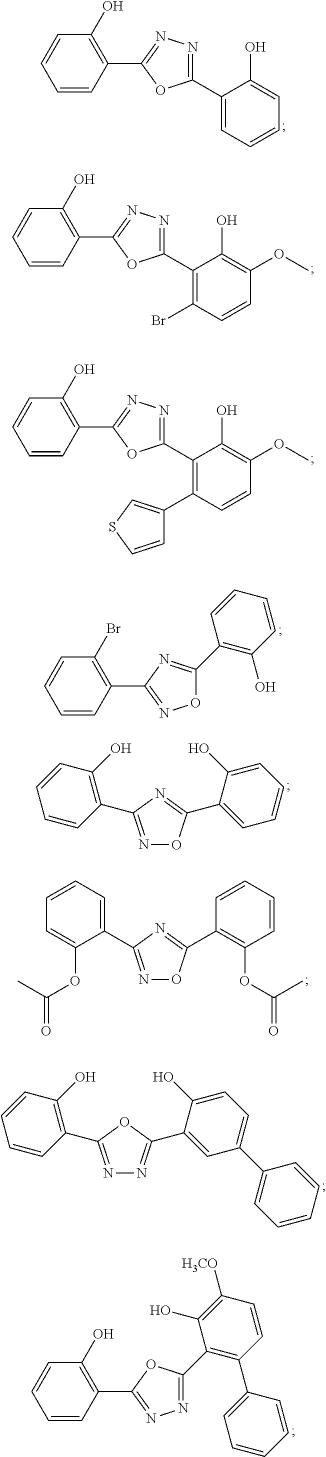 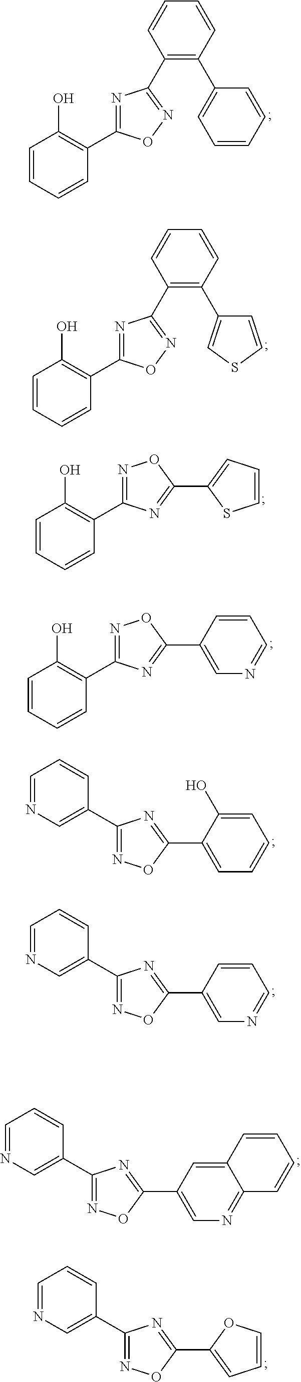 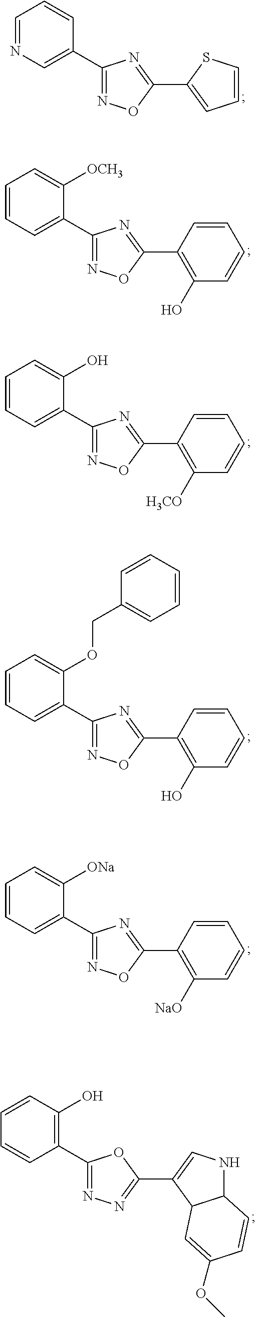 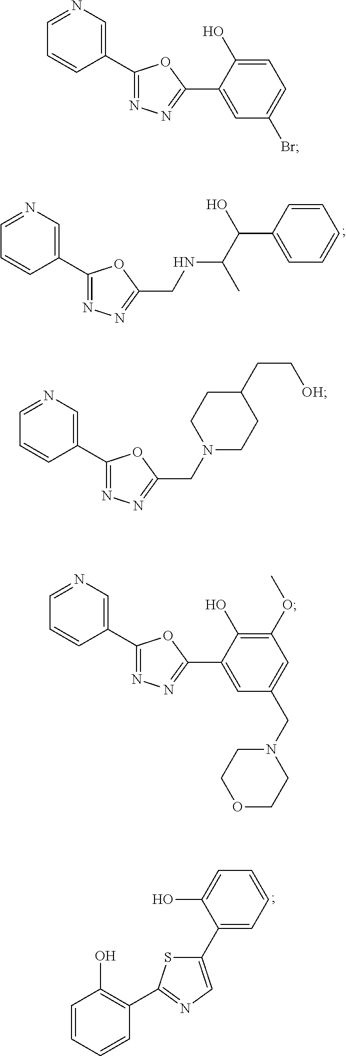 [SMILES: Oc1ccc(Br)cc1-c1nnc(-c2cccnc2)o1 ; CC(NCc1nnc(-c2cccnc2)o1)C(O)c1ccccc1 ; OCCC1CCN(Cc2nnc(-c3cccnc3)o2)CC1 ; COc1cc(CN2CCOCC2)cc(-c2nnc(-c3cccnc3)o2)c1O ; Oc1ccccc1-c1cnc(-c2ccccc2O)s1] 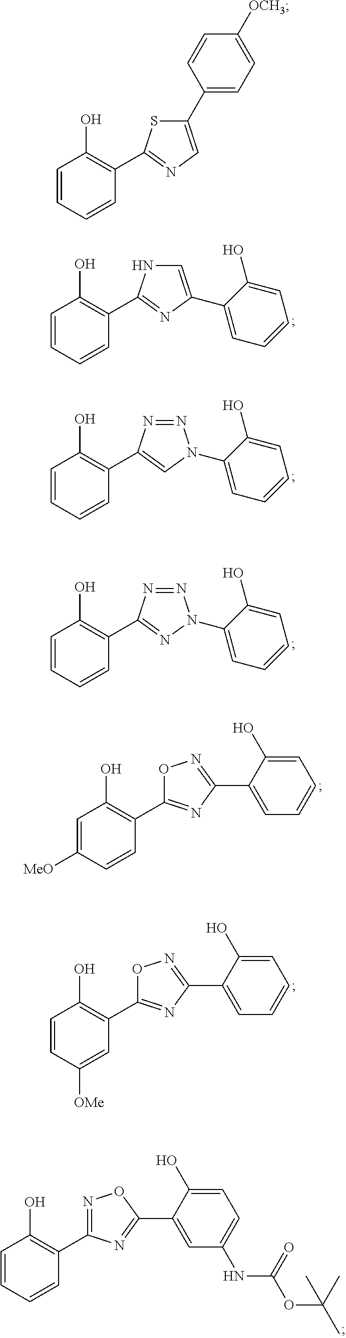 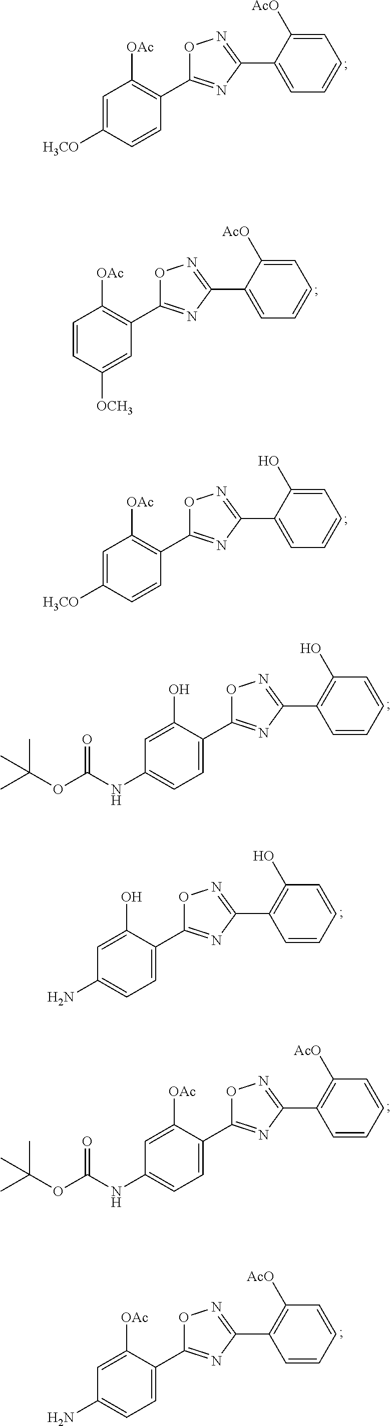 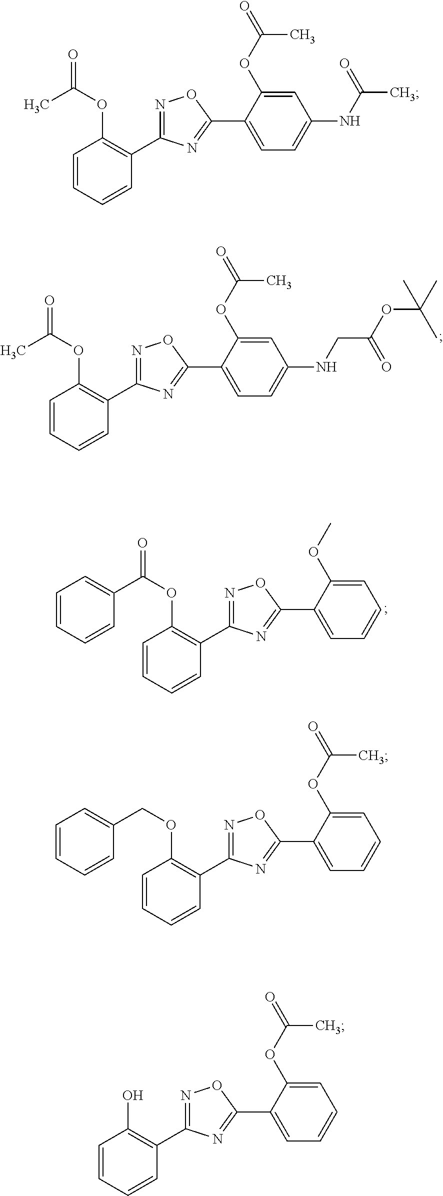 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]
a halogen substituted compound thereof; or a pharmaceutically acceptable salt, tautomer, or solvate thereof.

19. The method of claim 10, wherein the RRmod is an oxadiazole selected from: 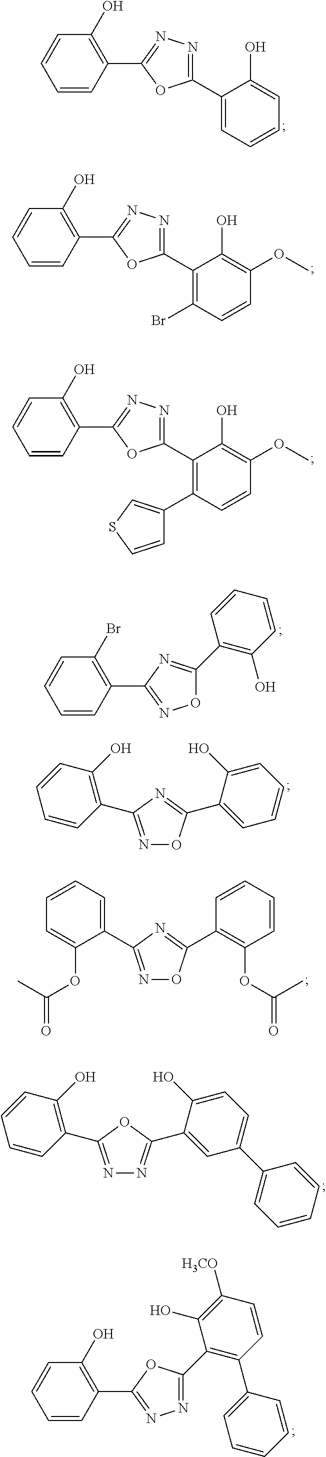 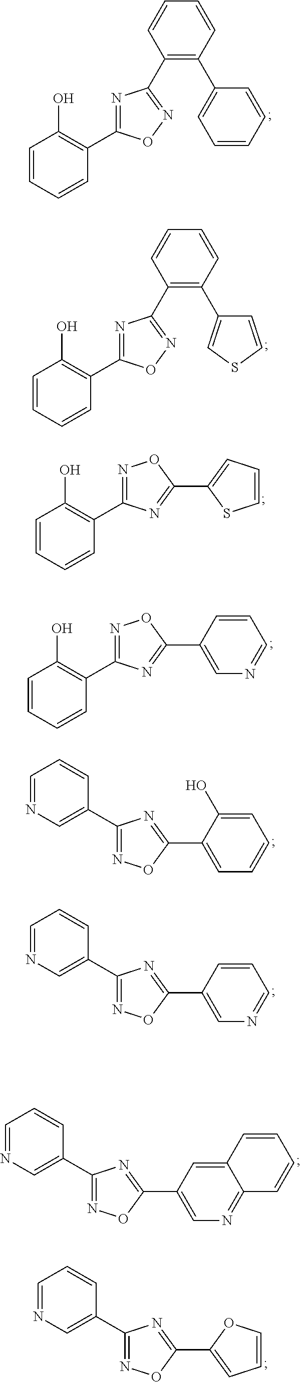 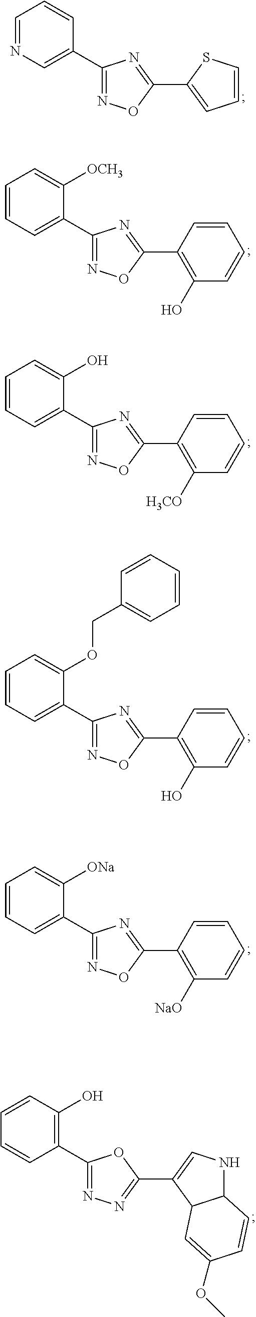 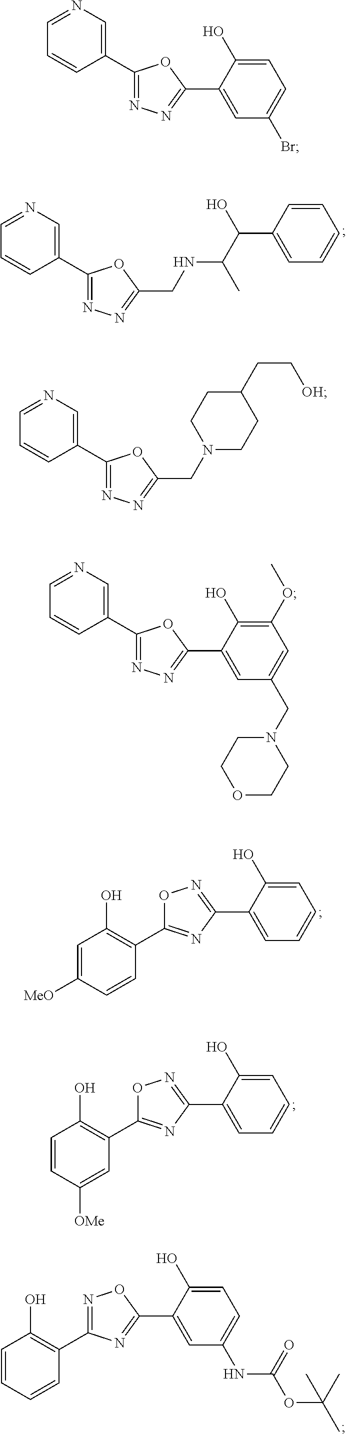 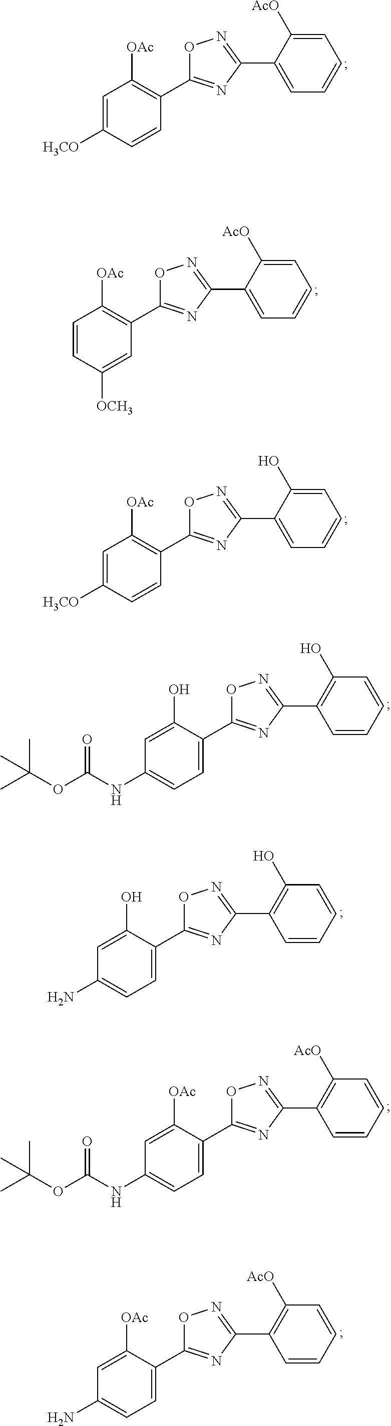 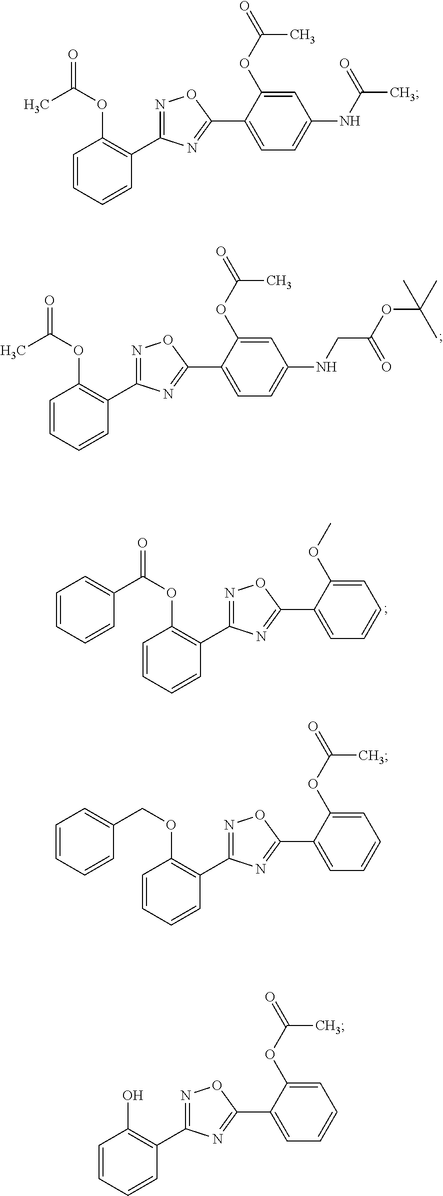 [SMILES: CC(=O)Nc1ccc(-c2nc(-c3ccccc3OC(C)=O)no2)c(OC(C)=O)c1 ; CC(=O)Oc1ccccc1-c1noc(-c2ccc(NCC(=O)OC(C)(C)C)cc2OC(C)=O)n1 ; COc1ccccc1-c1nc(-c2ccccc2OC(=O)c2ccccc2)no1 ; CC(=O)Oc1ccccc1-c1nc(-c2ccccc2OCc2ccccc2)no1 ; CC(=O)Oc1ccccc1-c1nc(-c2ccccc2O)no1]
a halogen substituted compound thereof; or a pharmaceutically acceptable salt, tautomer, or solvate thereof.

20. The method of claim 1, the cell comprising a cancer cell.
